(19) United States
(12) Patent Application Publication
(10) Pub. No.: US 2023/0416741 A1
(43) Pub. Date: Dec. 28, 2023

(54) METHOD OF TREATING DUCHENNE MUSCULAR DYSTROPHY

(71) Applicant: BioMarin Pharmaceutical Inc., Novato, CA (US)

(72) Inventors: Charles A. O'Neill, Sonoma, CA (US); Shripad S. Bhagwat, Solana Beach, CA (US); David Riley Neil, Philadelphia, PA (US); David Reid Jacoby, San Francisco, CA (US)

(21) Appl. No.: 18/311,789

(22) Filed: May 3, 2023

Related U.S. Application Data
(60) Provisional application No. 63/364,260, filed on May 5, 2022.
(60) Provisional application No. 63/387,733, filed on Dec. 16, 2022.

(51) Int. Cl. C12N 15/113; A61P 21/00

(52) U.S. Cl. CPC C12N 15/113; A61P 21/00; C12N 2310/11

(57) ABSTRACT

Provided herein are methods of treating or delaying the onset of Duchenne muscular dystrophy using modified antisense oligonucleotides.

### RELATED APPLICATIONS

This application claims the benefit of priority to U.S. provisional patent application Nos. 63/364260, filed May 5, 2022, and 63/387,733, filed Dec. 16, 2022. The disclosure of each of the above-referenced applications is incorporated by reference herein in its entirety.

### SEQUENCE LISTING

This application contains a Sequence Listing, which is being submitted herewith as an XML filed named “035105US.xml”, created on May 1, 2023, size 11,656 bytes, which is incorporated by reference herein in its entirety.

### FIELD

Provided herein are methods of treating or delaying the onset of Duchenne muscular dystrophy (DMD). In one embodiment, the methods include administration of a modified oligonucleotide to a subject having DMD.

### BACKGROUND

Antisense oligonucleotides (AONs) are in (pre)clinical development for many diseases and conditions, including cancer, inflammatory conditions, cardiovascular disease and neurodegenerative and neuromuscular disorders. Their mechanism of action is aimed at various targets, such as RNaseH-mediated degradation of target RNA in the nucleus or cytoplasm, at splice-modulation (exon inclusion or skipping) in the nucleus, or at translation inhibition by steric hindrance of ribosomal subunit binding in the cytoplasm. Splice-modulating or splice-switching AONs were first described for correction of aberrant splicing in human β-globin pre-mRNAs (Dominski and Kole PNAS, 1993, 90(18):8673-8677), and are currently being studied for a variety of genetic disorders.

AONs have been extensively studied in the treatment of the neuromuscular disorders Duchenne muscular dystrophy (DMD) and Becker muscular dytrophy (BMD). Duchenne muscular dystrophy (DMD) and Becker muscular dystrophy (BMD) are the most common childhood forms of muscular dystrophy. DMD is a severe, lethal neuromuscular disorder resulting in a dependency on wheelchair support before the age of 12 and patients often die before the age of thirty due to respiratory or heart failure. It is caused by reading frame-shifting deletions (˜67%) or duplications (˜7%) of one or more exons, or by point mutations (˜25%) in the 2.24 Mb dystrophin gene, resulting in the absence of functional dystrophin. BMD is also caused by mutations in the dystrophin gene, but these maintain the open reading frame, yield semi-functional dystrophin proteins, and result in a typically much milder phenotype and longer lifespan.

To date, four AONs have been approved for treatment of DMD: eteplirsen, golodirsen, casimersen and viltolarsen. However, these drugs possess limited efficacy and are each approved for treating only a small percentage of DMD patients.

Thus, there is a continuing need for methods of treating DMD.

### SUMMARY

Provided herein are methods of treating or delaying the onset of DMD by administering a modified oligonucleotide to a subject having DMD. In one embodiment, the modified oligonucleotide is an antisense oligonucleotide (AON).

### BRIEF DESCRIPTION OF THE DRAWINGS

FIG. 1 shows the plasma concentration of AON1 over time.

FIG. 2 shows the AON1 concentration in heart, quadriceps, liver, diaphragm, gastrocnemius and kidney tissue.

FIG. 3 shows that AON1 achieved sustained dose dependent exon 51 skipping in muscle tissue (quadriceps, gastrocnemius, heart and diaphragm) with 25 weeks of treatment.

FIG. 4 shows dystrophin wt % in heart and quadriceps and shows Overall Gait Score for mice treated with AON1.

FIG. 5 shows that AON1 treatment attenuates hDMD del52(+/+) model-associated muscle pathology and sustains pharmcological effect for 12 weeks post-25 week dosing.

FIG. 6 shows that AON1 treatment attenuates hDMD del52(+/+) model-associated cardiac pathology and sustains pharmacological effect for 12 weeks.

FIG. 7 shows dystrophin expression in heart and quadriceps of hDMDΔ52/mdx mice treated with the AON1 having the TEG linked to the oligonucleotide by a thiophosphate group (AON1 PS, 9.4, 18.7 or 37.5 mg/kg) at 14 (solid circles) or 28 days (open circles) post-dosing QW, Q2W or Q4W.

FIG. 8 shows % exon 51 skipping in heart of hDMDΔ52/mdx mice treated with AON1 PS (9.4, 18.7 or 37.5 mg/kg) at 14 (solid circles) or 28 days (open circles) post-dosing QW, Q2W or Q4W.

FIG. 9 shows % exon 51 skipping in quadriceps, gastrocnemius, heart and diaphragm of hDMDA52/mdx mice treated with 18.7 mg/kg AON1 (13 weeks QW) at 4 and 8 weeks post-dosing.

FIG. 10 shows dystrophin expression in heart and quadriceps of hDMDΔ52/mdx mice treated with the AON1 (18.7 mg/kg) at 4 or 8 weeks post-dosing QWx13W.

FIG. 11 shows Overall Gait Score for mice treated with AON1.

FIG. 12 shows the effect of AON1 PS and AON2 (AON1 without the 5′-TEG group) on complement parameter Bb in human and monkey serum.

FIG. 13 shows the effect of AON1 PS and AON2 on complement parameter C3a in human and monkey serum.

FIG. 14 shows exon skipping in biceps, gastrocnemius and heart tissue for NHPs treated for 39 weeks with AON1 (Example 8).

### DETAILED DESCRIPTION

### I. DDEFINITIONS

To facilitate understanding of the disclosure set forth herein, a number of terms are defined below.

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

The singular forms “a,” “an,” and “the” include plural references, unless the context clearly dictates otherwise.

As used herein “subject” is an animal, such as a mammal, including human, such as a patient.

As used herein, biological activity refers to the in vivo activities of a compound or physiological responses that result upon in vivo administration of a compound, composition or other mixture. Biological activity, thus, encompasses therapeutic effects and pharmacokinetic behavior of such compounds, compositions and mixtures. Biological activities can be observed in in vitro systems designed to test for such activities.

As used herein, treatment means any manner in which one or more of the symptoms of a disease or disorder are ameliorated or otherwise beneficially altered. Treatment also encompasses any pharmaceutical use of the compositions herein, such as use for treating DMD.

As used herein, amelioration of the symptoms of a particular disorder by administration of a particular compound or pharmaceutical composition refers to any lessening, whether permanent or temporary, lasting or transient that can be attributed to or associated with administration of the compound or pharmaceutical composition.

As used herein, and unless otherwise indicated, the terms “manage,” “managing” and “management” encompass preventing the recurrence of the specified disease or disorder in a subject who has already suffered from the disease or disorder, and/or lengthening the time that a subject who has suffered from the disease or disorder remains in remission. The terms encompass modulating the threshold, development and/or duration of the disease or disorder, or changing the way that a subject responds to the disease or disorder.

### II. AONS FOR USE IN THE METHODS PROVIDED HEREIN

In one embodiment, the AONs for use in the methods provided herein are reverse complementary to a portion of exon 51 of human dystrophin pre-mRNA. In another embodiment, the AONs for use in the methods provided herein have the sequence 5′-gguaaguucuguccaagc-3′ (SEQ ID NO: 1) and contain a modification. In another embodiment, the AONs for use in the methods provided herein have the sequence 5′-gguaaguuc*uguc*c*aagc*-3′ (SEQ ID NO: 2), where c* is 5-methylcytosine and g and c* are LNA nucleotides. As used herein, an LNA nucleotide has the structure:

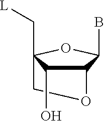

where B is a nucleobase and L is phosphate or phosphorothioate linkage to another nucleotide.

In another embodiment, the AON for use in the methods provided herein has the sequence TEG-5′-gguaaguuc*uguc*c*aagc*-3′, where c* is 5-methylcytosine, g and c* are LNA nucleotides, and TEG is a tri-ethylene glycol group. In another embodiment, the AON for use in the methods provided herein has the sequence TEG-5′-gguaaguuc*uguc*c*aagc*-3′, where c* is 5-methylcytosine, g and c* are LNA nucleotides, TEG is a tri-ethylene glycol group attached to the 5′ terminus via a phosphate group, the internucleoside linkages are phosphorothioate linkages, and the non-LNA nucleotides are 2′-OMe nucleotides (referred to herein as “AON1”). See WO 2022/069511 A1 and US 2022/0098586 A1.

### III. METHODS OF TREATING DMD

Provided herein are methods of treating or delaying the onset of DMD by administering a modified oligonucleotide to a subject having DMD. In one embodiment, the modified oligonucleotide is an antisense oligonucleotide (AON). In another embodiment, the AON is an AON disclosed herein. In another embodiment, the AON is AON1. In another embodiment, provided is a method of treating DMD by administering AON1 to a subject having DMD. In another embodiment, provided is a method of delaying the onset of DMD by administering AON1 to a subject having DMD.

In certain embodiments, the AON is administered in a dose of 0.4 mg/kg, 0.6 mg/kg, 0.8 mg/kg, 1.5 mg/kg, 3 mg/kg, 6 mg/kg, 9 mg/kg, 12 mg/kg or 18 mg/kg. In another embodiment, the AON is administered QW. In another embodiment, the AON is administered QWx15, 16, 17, 18, 19, 20, 21, 22, 23, 24, 25, 26, 27, 28, 29, 30, 31, 32, 33, 34 or 35W. In another embodiment, the AON is administered QWx24W or QWx25W. In another embodiment, the AON is administered QWx25W. In another embodiment, the AON is administered QWx24W.

In certain embodiments, AON1 is administered in a dose of 0.4 mg/kg, 0.6 mg/kg, 0.8 mg/kg, 1.5 mg/kg, 3 mg/kg, 6 mg/kg, 9 mg/kg, 12 mg/kg or 18 mg/kg. In another embodiment, AON1 is administered QW. In another embodiment, AON1 is administered QWx15, 16, 17, 18, 19, 20, 21, 22, 23, 24, 25, 26, 27, 28, 29, 30, 31, 32, 33, 34 or 35W. In another embodiment, AON1 is administered QWx24W or QWx25W. In another embodiment, AON1 is administered QWx25W. In another embodiment, AON1 is administered QWx24W.

In another embodiment, AON1 is administered 6 mg/kg QWx25W. In another embodiment, AON1 is administered 18 mg/kg QWx25W.

In another embodiment, provided is a method of treating DMD by administering AON1 at 6 mg/kg QWx24W. In another embodiment, provided is a method of treating DMD by administering AON1 at 18 mg/kg QWx24W. In another embodiment, provided is a method of delaying the onset of DMD by administering AON1 at 6 mg/kg QWx24W. In another embodiment, provided is a method of delaying the onset of DMD by administering AON1 at 18 mg/kg QWx24W.

In certain embodiments, the methods provided herein result in superior outcomes as compared to prior methods of treating or delaying the onset of DMD. In one embodiment, the methods provided herein induce changes toward normalization in relevant disease modifying biomarkers. In another embodiment, the methods provided herein induce changes toward normalization in disease related clinical and anatomical muscle pathology. In another embodiment, the methods provided herein give partial to near complete rescue of DMD phenotype as measured in the MotoRater/Gait Score. In another embodiment, the methods provided herein give increased dystrophin production. In another embodiment, the methods provided herein give increased skipping of exon 51 of human dystrophin pre-mRNA. In another embodiment, the methods alleviate one or more symptom(s) of DMD.

Alleviating one or more symptom(s) of DMD in an individual using an AON provided herien may be assessed by any of the following assays: prolongation of time to loss of walking, improvement of muscle strength, improvement of the ability to lift weight, improvement of the time taken to rise from the floor, improvement in the nine-meter walking time, improvement in the time taken for four-stairs climbing, improvement of the leg function grade, improvement of the pulmonary function, improvement of cardiac function, improvement of the quality of life. Each of these assays is known to the skilled person. For each of these assays, as soon as a detectable improvement or prolongation of a parameter measured in an assay has been found, it will generally mean that one or more symptoms of DMD has been alleviated in an individual using an AON provided herein. Detectable improvement or prolongation is in one embodiment a statistically significant improvement or prolongation as described in Hodgetts et al. Neuromuscular Disorders 2006; 16: 591-602. Alternatively, the alleviation of one or more symptom(s) of DMD may be assessed by measuring an improvement of a muscle fiber function, integrity and/or survival. In another embodiment, one or more symptom(s) of a DMD patient is/are alleviated and/or one or more characteristic(s) of one or more muscle cells from a DMD patient is/are improved. Such symptoms or characteristics may be assessed at the cellular, tissue level or on the patient self

An alleviation of one or more characteristics of a muscle cell from a patient may be assessed by any of the following assays on a myogenic cell or muscle cell from a patient: reduced calcium uptake by muscle cells, decreased collagen synthesis, altered morphology, altered lipid biosynthesis, decreased oxidative stress, and/or improved muscle fiber function, integrity, and/or survival. These parameters are usually assessed using immunofluorescence and/or histochemical analyses of cross sections of muscle biopsies.

The improvement of muscle fiber function, integrity and/or survival may be assessed using at least one of the following assays: a detectable decrease of creatine kinase in blood, a detectable decrease of necrosis of muscle fibers in a biopsy cross-section of a muscle suspected to be dystrophic, and/or a detectable increase of the homogeneity of the diameter of muscle fibers in a biopsy cross-section of a muscle suspected to be dystrophic. Each of these assays is known to the skilled person.

Creatine kinase may be detected in blood as described in Hodgetts et al. (2006). A detectable decrease in creatine kinase may mean a decrease of 5%, 10%, 20%, 30%, 40%, 50%, 60%, 70%, 80%, 90% or more compared to the concentration of creatine kinase in a same DMD patient before treatment.

A detectable decrease of necrosis of muscle fibers is generally assessed in a muscle biopsy, such as described in Hodgetts et al. (2006), using biopsy cross-sections. A detectable decrease of necrosis may be a decrease of 5%, 10%, 20%, 30%, 40%, 50%, 60%, 70%, 80%, 90% or more of the area wherein necrosis has been identified using biopsy cross-sections. The decrease is measured by comparison to the necrosis as assessed in a same DMD patient before treatment.

A detectable increase of the homogeneity of the diameter of a muscle fiber is generally assessed in a muscle biopsy cross-section, such as described in Hodgetts et al. (supra). The increase is measured by comparison to the homogeneity of the diameter of a muscle fiber in a same DMD patient before treatment.

In one embodiment, an AON provided herein provides said individual with a functional or a semi-functional dystrophin protein, and is able to, for at least in part decrease the production of an aberrant dystrophin protein in said individual.

In one embodiment, providing an individual with a functional or a semi-functional dystrophin protein means an increase in the production of functional or semi-functional dystrophin protein. Increasing the production of functional or semi-functional dystrophin mRNA, or functional or semi-functional dystrophin protein, means a detectable increase or at least 5%, 10%, 20%, 30%, 40%, 50%, 60%, 70%, 80%, 90%, 100%, 120%, 140%, 160%, 180%, 200% or more compared to the initial amount of functional or semi-functional mRNA, or functional or semi-functional dystrophin protein, as detectable by RT- digital droplet PCR (mRNA) (Verheul et al., PLoS ONE 2016, 11(9):e0162467) or immunofluorescence (Beekman et al., PLoS ONE 2014; 9(9): e107494), western blot, or capillary Western immunoassay (Beekman et al., PLoS ONE 2018; 13(4): e0195850) analysis (protein). In another embodiment, said initial amount is the amount of functional or semifunctional mRNA, or functional or semi-functional dystrophin protein, at the onset of inducing exon-skipping in the dystrophin pre-mRNA in a cell, in an organ, in a tissue and/or in an individual using a compound of the invention.

Decreasing the production of an aberrant dystrophin mRNA, or aberrant dystrophin protein, means that 90%, 80%, 70%, 60%, 50%, 40%, 30%, 20%, 10%, 5% or less of the initial amount of aberrant dystrophin mRNA, or aberrant dystrophin protein, is still detectable by RT-digital droplet PCR (mRNA) or immunofluorescence, western blot, or capillary Western immunoassay (Wes) analysis (protein). In one embodiment, said initial amount is the amount of aberrant dystrophin mRNA, or aberrant dystrophin protein, at the onset of inducing exon-skipping in the dystrophin pre-mRNA in a cell, in an organ, in a tissue and/or in an individual using an AON of the invention. An aberrant dystrophin mRNA or protein is also referred to herein as a less functional (compared to a wild type functional dystrophin protein) or a non-functional dystrophin mRNA or protein. A non-functional dystrophin protein is a dystrophin protein which is not able to bind actin and/or members of the DGC protein complex. A non-functional dystrophin protein or dystrophin mRNA does typically not have or does not encode a dystrophin protein with an intact C-terminus of the protein. The detection of a functional or semi-functional dystrophin mRNA or protein may be done as for an aberrant dystrophin mRNA or protein.

Once a DMD patient is provided with a functional or a semi-functional dystrophin protein, at least part of the cause of DMD is taken away. Hence, it would then be expected that the symptoms of DMD are at least partly alleviated, or that the rate with which the symptoms worsen is decreased, resulting in a slower decline. The enhanced skipping frequency also increases the level of functional or a semi-functional dystrophin protein produced in a muscle cell of a DMD individual.

### IV. PHARMACEUTICAL COMPOSITIONS FOR USE IN THE METHODS

The pharmaceutical compositions provided herein contain therapeutically effective amounts of one or more of the AONs provided herein and a pharmaceutically acceptable carrier, diluent or excipient.

The AONs can be formulated into suitable pharmaceutical preparations such as solutions, suspensions, powders, in sterile solutions or suspensions for ophthalmic or parenteral administration, as well as transdermal patch preparation. Typically, the AONs described herein are formulated into pharmaceutical compositions using techniques and procedures well known in the art (see, e.g., Ansel Introduction to Pharmaceutical Dosage Forms, Seventh Edition 1999).

In the compositions, effective concentrations of one or more compounds or pharmaceutically acceptable salts is (are) mixed with a suitable pharmaceutical carrier or vehicle. In certain embodiments, the concentrations of the AONs in the compositions are effective for delivery of an amount, upon administration, that treats, prevents, or ameliorates one or more of the symptoms and/or progression of a disease or disorder disclosed herein.

Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the AONs provided herein include any such carriers known to those skilled in the art to be suitable for the particular mode of administration.

In addition, the AONs may be formulated as the sole pharmaceutically active ingredient in the composition or may be combined with other active ingredients. Liposomal suspensions, including tissue-targeted liposomes, may also be suitable as pharmaceutically acceptable carriers. These may be prepared according to methods known to those skilled in the art. For example, liposome formulations may be prepared as known in the art. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The active compound is included in the pharmaceutically acceptable carrier in an amount sufficient to exert a therapeutically useful effect in the absence of undesirable side effects on the subject treated. The therapeutically effective concentration may be determined empirically by testing the AONs in in vitro and in vivo systems described herein and then extrapolated therefrom for dosages for humans. In some embodiments, the AON is administered in a method to achieve a therapeutically effective concentration of the drug. In some embodiments, a companion diagnostic (see, e.g., Olsen D and Jorgensen J T, Front. Oncol., 2014 May 16, 4:105, doi: 10.3389/fonC.2014.00105) is used to determine the therapeutic concentration and safety profile of the active compound in specific subjects or subject populations.

The concentration of AON in the pharmaceutical composition will depend on absorption, tissue distribution, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of a disease or disorder disclosed herein.

In certain embodiments, a therapeutically effective dosage should produce a serum concentration of active ingredient of from about 0.1 ng/mL to about 50-100 μg/mL. In one embodiment, the pharmaceutical compositions provide a dosage of from about 0.001 mg to about 2000 mg of compound per kilogram of body weight per day. Pharmaceutical dosage unit forms are prepared to provide from about 1 mg to about 1000 mg and in certain embodiments, from about 10 to about 500 mg of the essential active ingredient or a combination of essential ingredients per dosage unit form.

The AON may be administered at once or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Thus, effective concentrations or amounts of one or more of the AONs described herein or pharmaceutically acceptable salts thereof are mixed with a suitable pharmaceutical carrier or vehicle for systemic, topical or local administration to form pharmaceutical compositions. AONs are included in an amount effective for ameliorating one or more symptoms of, or for treating, retarding progression, or preventing. The concentration of active compound in the composition will depend on absorption, tissue distribution, inactivation, excretion rates of the active compound, the dosage schedule, amount administered, particular formulation as well as other factors known to those of skill in the art.

The compositions are intended to be administered by a suitable route, including but not limited to parenteral, subcutaneous, intravenous, intramuscular, intraperitoneal, intrathecal, dermal, transdermal or buccal.

Solutions or suspensions used for parenteral, intradermal or subcutaneous application can include any of the following components: a sterile diluent, such as water for injection, saline solution, fixed oil, polyethylene glycol, glycerin, propylene glycol, dimethyl acetamide or other synthetic solvent; antimicrobial agents, such as benzyl alcohol and methyl parabens; antioxidants, such as ascorbic acid and sodium bisulfate; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, pens, disposable syringes or single or multiple dose vials made of glass, plastic or other suitable material.

In instances in which the AONs exhibit insufficient solubility, methods for solubilizing AONs may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the AON(s), the resulting mixture may be a solution, suspension, emulsion or the like. The form of the resulting mixture depends upon a number of factors, including the intended mode of administration and the solubility of the AON in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as powders, granules, sterile parenteral solutions or suspensions, and oil water emulsions containing suitable quantities of the AONs or pharmaceutically acceptable salts thereof. The pharmaceutically therapeutically active AONs and salts thereof are formulated and administered in unit dosage forms or multiple dosage forms. Unit dose forms as used herein refer to physically discrete units suitable for human and animal subjects and packaged individually as is known in the art. Each unit dose contains a predetermined quantity of the therapeutically active compound sufficient to produce the desired therapeutic effect, in association with the required pharmaceutical carrier, vehicle or diluent. Examples of unit dose forms include ampules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses which are not segregated in packaging.

Sustained-release preparations can also be prepared. Suitable examples of sustained-release preparations include semipermeable matrices of solid hydrophobic polymers containing the compound provided herein, which matrices are in the form of shaped articles, e.g., films, or microcapsule. Examples of sustained-release matrices include iontophoresis patches, polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(-)-3-hydroxybutyric acid. While polymers such as ethylene-vinyl acetate and lactic acid-glycolic acid enable release of molecules for over 100 days, certain hydrogels release proteins for shorter time periods. When encapsulated compound remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Dosage forms or compositions containing active ingredient in the range of 0.005% to 100% with the balance made up from non-toxic carrier may be prepared. Such compositions include solutions, suspensions, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain about 0.001% 100% active ingredient, in certain embodiments, about 0.1 85% or about 75-95%.

The active AONs or pharmaceutically acceptable salts may be prepared with carriers that protect the compound against rapid elimination from the body, such as time release formulations or coatings.

The compositions may include other active AONs to obtain desired combinations of properties. The AONs provided herein, or pharmaceutically acceptable salts thereof as described herein, may also be advantageously administered for therapeutic or prophylactic purposes together with another pharmacological agent known in the general art to be of value in treating one or more of the diseases or medical conditions referred to hereinabove, such as diseases related to oxidative stress. It is to be understood that such combination therapy constitutes a further aspect of the compositions and methods of treatment provided herein.

### A. Injectables, Solutions and Emulsions

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. In some embodiments, the suspension is a suspension of microparticles or nanoparticles. In some embodiments, the emulsion is an emulsion of microparticles or nanoparticles. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow release or sustained release system, such that a constant level of dosage is maintained is also contemplated herein. Briefly, an AON provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The AON diffuses through the outer polymeric membrane in a release rate controlling step. The percentage of active AON contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple dose containers which include phenols or cresols, mercurials, benzyl alcohol, chlorobutanol, methyl and propyl phydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active AON is adjusted so that an injection provides an effective amount to produce the desired pharmacological effect. The exact dose depends on the age, weight and condition of the subject or animal as is known in the art.

The unit dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active AON is an effective mode of administration. Another embodiment is a sterile aqueous or oily solution or suspension containing an active AON injected as necessary to produce the desired pharmacological effect.

Injectables are designed for local and systemic administration. Typically, a therapeutically effective dosage is formulated to contain a concentration of at least about w/w up to about 90% w/w or more, such as more than 1% w/w of the active AON to the treated tissue(s). The active AON may be administered at once or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The AON may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

### B. Lyophilized Powders

Also provided herein are lyophilized powders, which can be reconstituted for administration as solutions, emulsions and other mixtures. They may also be reconstituted and formulated as solids or gels.

The sterile, lyophilized powder is prepared by dissolving an AON provided herein, or a pharmaceutically acceptable salt thereof, in a suitable solvent. The solvent may contain an excipient which improves the stability or other pharmacological component of the powder or reconstituted solution, prepared from the powder. Excipients that may be used include, but are not limited to, dextrose, sorbitol, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (including but not limited to 10-1000 mg or 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, about 5-mg, or about 9-30 mg of lyophilized powder, is added per mL of sterile water or other suitable carrier. The precise amount depends upon the selected compound. Such amount can be empirically determined.

### C. Topical Administration

Topical mixtures are prepared as described for the local and systemic administration. The resulting mixture may be a solution, suspension, emulsion or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

### D. Sustained Release Compositions

AONs provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; 4,008,719, 5,674,533, 5,059,595, 5,591,767, 5,120,548, 5,639,476, 5,354,556, 5,639,480, 5,733,566, 5,739,108, 5,891,474, 5,922,356, 5,980,945, 5,993,855, 6,045,830, 6,087,324, 6,113,943, 6,197,350, 6,248,363, 6,264,970, 6,267,981, 6,376,461, 6,419,961, 6,589,548, 6,613,358, 6,699,500 and 6,740,634, each of which is incorporated herein by reference. Such dosage forms can be used to provide slow or controlled-release of one or more active ingredients using, for example, hydroxypropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. In one embodiment, the use of an optimally designed controlled-release preparation in medical treatment is characterized by a minimum of drug substance being employed to cure or control the condition in a minimum amount of time. In certain embodiments, advantages of controlled-release formulations include extended activity of the drug, reduced dosage frequency, and increased subject compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled release of an AON can be stimulated by various conditions including, but not limited to, pH, temperature, enzymes, water, or other physiological conditions or compounds.

In certain embodiments, the AON may be administered using intravenous infusion, an implantable osmotic pump, a transdermal patch, liposomes, or other modes of administration. In one embodiment, a pump may be used (see, Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity of the therapeutic target, i.e., thus requiring only a fraction of the systemic dose (see, e.g., Goodson, Medical Applications of Controlled Release, vol. 2, pp. 115-138 (1984).

Other controlled release systems are discussed in the review by Langer (Science 249:1527-1533 (1990). The AON can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The AON then diffuses through the outer polymeric membrane in a release rate controlling step. The percentage of active ingredient contained in such parenteral compositions is highly dependent on the specific nature thereof, as well as the needs of the subject.

### E. Targeted Formulations

The AONs provided herein, or pharmaceutically acceptable salts thereof, may also be formulated to be targeted to a particular tissue, receptor, or other area of the body of the subject to be treated, including liposome-, resealed erythrocyte-, and antibody-based delivery systems. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, the antibody-based delivery system is an antibody-drug conjugate (“ADC”), e.g., as described in Hamilton G S, Biologicals, 2015 September, 43(5):318-32; Kim E G and Kim K M, Biomol. Ther. (Seoul), 2015 Nov., 23(6):493-509; and Peters C and Brown S, Biosci. Rep., 2015 Jun. 12, 35(4) pii: e00225, each of which is incorporated herein by reference.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of an AON provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

### F. Articles of Manufacture

The AONs or pharmaceutically acceptable salts can be packaged as articles of manufacture containing packaging material, an AON or pharmaceutically acceptable salt thereof provided herein, which is used for treatment, prevention or amelioration of one or more symptoms or progression of a disease or disorder disclosed herein, and a label that indicates that the compound or pharmaceutically acceptable salt thereof is used for treatment, prevention or amelioration of one or more symptoms or progression of a disease or disorder disclosed herein.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, pens, bottles, and any packaging material suitable for a selected formulation and intended mode of administration and treatment. A wide array of formulations of the AONs and compositions provided herein are contemplated.

In certain embodiments, provided herein also are kits which, when used by the medical practitioner, can simplify the administration of appropriate amounts of AONs to a subject. In certain embodiments, the kit provided herein includes a container and a dosage form of an AON provided herein or a pharmaceutically acceptable salt, solvate, or prodrug thereof

In certain embodiments, the kit includes a container comprising a dosage form of the AON provided herein or a pharmaceutically acceptable salt, solvate, or prodrug thereof, in a container comprising one or more other therapeutic agent(s) described herein.

Kits provided herein can further include devices that are used to administer the AONs. Examples of such devices include, but are not limited to, syringes, needle-less injectors drip bags, patches, and inhalers.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

### VII. COMBINATION THERAPY WITH A SECOND ACTIVE AGENT

Also provided herein are methods of combination therapy using an AON disclosed herein with other therapeutic agents useful in treating or delaying the onset of DMD. In another embodiment, provided herein are methods of combination therapy using AON1 with other therapeutic agents useful in treating or delaying the onset of DMD. In these methods, the AON disclosed herein, e.g., AON1, is administered as described elsewhere herein, e.g., QWx24W.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as an AON provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

Administration of the AON disclosed herein and one or more second active agents to a subject can occur simultaneously or sequentially by the same or different routes of administration. The suitability of a particular route of administration employed for a particular active agent will depend on the active agent itself (e.g., whether it can be administered orally without decomposing prior to entering the blood stream) and the disease or disorder being treated.

The route of administration of the AON disclosed herein is independent of the route of administration of a second therapy. In another embodiment, the AON disclosed herein is administered intravenously. Thus, in accordance with these embodiments, the AON disclosed herein is administered intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. In one embodiment, the AON disclosed herein and a second therapy are administered by the same mode of administration, e.g., by IV. In another embodiment, the AON disclosed herein is administered by one mode of administration, e.g., by IV, whereas the second agent is administered by another mode of administration, e.g., orally.

In one embodiment, the second active agent is administered intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the type of disease being treated or managed, the severity and stage of disease, and the amount of the AON provided herein, or a derivative thereof, and any optional additional active agents concurrently administered to the subject.

One or more second active ingredients or agents can be used together with the AON provided herein, or a derivative thereof, in the methods and compositions provided herein. Second active agents can be large molecules (e.g., proteins) or small molecules (e.g., synthetic inorganic, organometallic, or organic molecules).

Examples of large molecule active agents include, but are not limited to, hematopoietic growth factors, cytokines, AONs and monoclonal and polyclonal antibodies. Typical large molecule active agents are biological molecules, such as naturally occurring or synthetic or recombinant proteins. Specific examples of AONs for use herein as second active agents include eteplirsen, casimersen, golodirsen, viltolarsen and SRP-5051 (Sarepta Therapeutics).

Examples of small molecules include corticosteroids, such as deflazacort.

Other therapies that may be combined with the AONs disclosed herein include gene therapy (e.g., SRP-9001, GALGT2 or GNT 0004 (Sarepta Therapeutics)), gene editing (e.g., CRISPR/CAS9 (Sarepta Therapeutics)) and cellular therapy (e.g., CAP-1002 (Capricor Therapeutics/Nippon Shinyaku Co. Ltd.)).

### VIII. EXAMPLES

The examples below are meant to illustrate certain embodiments provided herein, and not to limit the scope of this disclosure.

### Example 1

### AON1 Administered for 25 Weeks in de152hDMD/mdx Mice

hDMD de152/mdx (+/+) and C57BL/6J wild-type (WT) mice (˜7 weeks of age on Day 1) were divided into four Groups and dosed as shown below:

| | | | | | | | Termination | |
|---|---|---|---|---|---|---|---|---|
| | | | | | | | N (Post- | |
| | | | | | Dose | Dose | Final Dose) | |
| | | # of Mice | Dose | IV Dose | Level | Volume | 4 | 12 |
| Group | Strain | (M/F/Mix) | Article | Regimen | (mg/kg) | (mL/kg) | Wks | Wks |
| 1 | WT | 18 | Vehicle | QWx25W | 0 | 8 | 6 | 12 |
| 2 | del52/mdx | 18 | Vehicle | QWx25W | 0 | 8 | 6 | 12 |
| 3 | del52/mdx | 18 | AON1 | QWx25W | 6 | 8 | 6 | 12 |
| 4 | del52/mdx | 18 | AON1 | QWx25W | 18 | 8 | 6 | 12 |

The dose of 6 mg/kg/dose equals 0.92 μmol/kg/dose (MW of AON1 is 6517 g/mol). The dose of 18 mg/kg/dose equals 2.76 μmol/kg/dose. Thus, the total amount of AON1 dosed over 25 W is 150 mg/kg (23.00 μmol/kg) for the 6 mg/kg dose level and 450 mg/kg (69.00 μmol/kg) for the 18 mg/kg dose level.

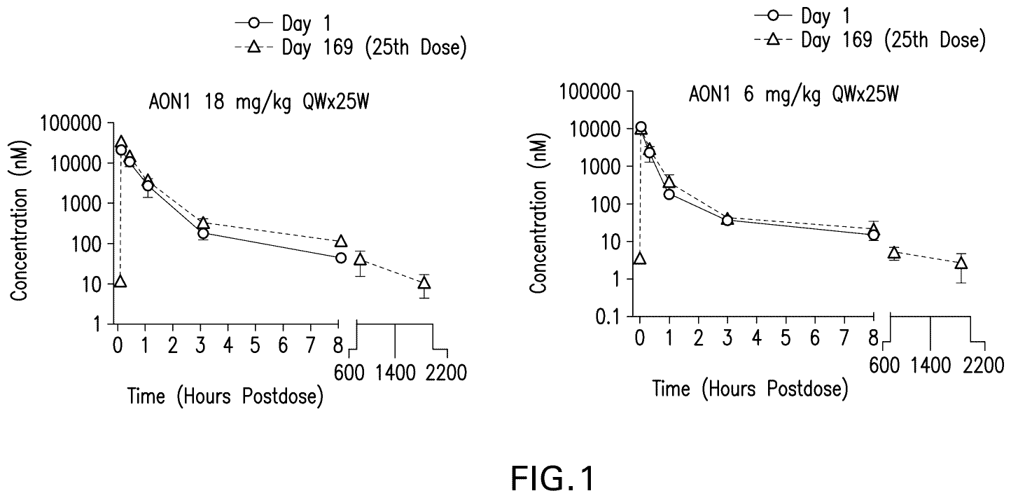

Post-dosing, the plasma concentration, tissue distribution, concentration, exon skipping, overall Gait Score, and cellular protection were analyzed. Results are shown in FIGS. 1-6.

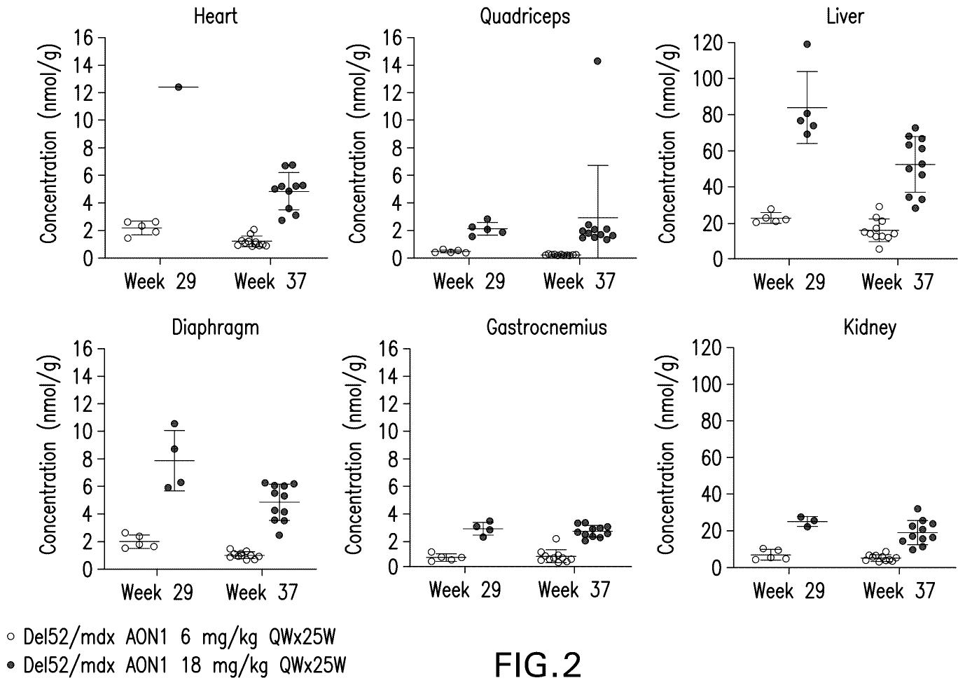

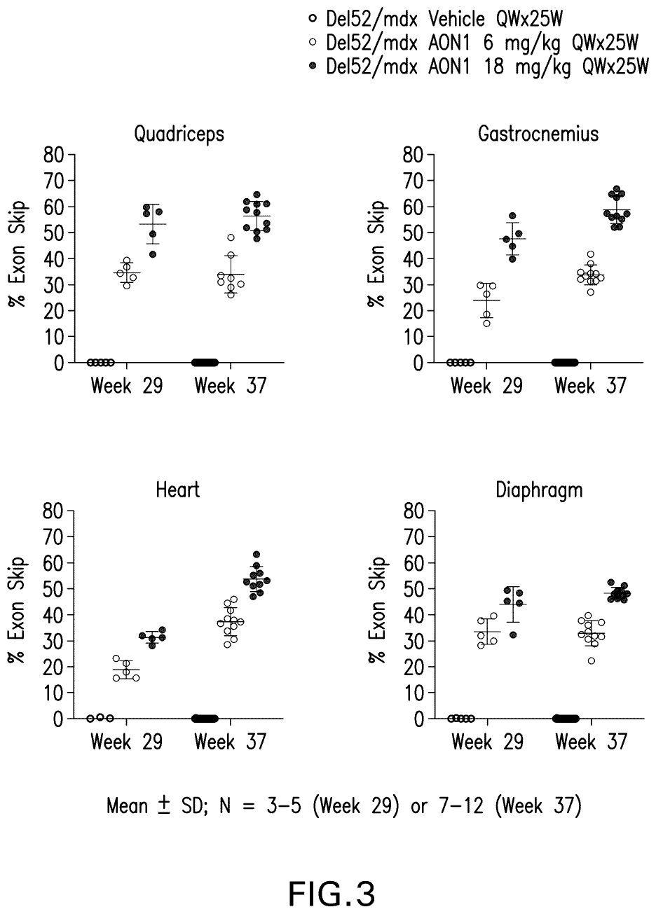

Briefly, FIG. 1 shows the plasma concentration of AON1 over time. FIG. 2 shows the AON1 concentration in heart, quadriceps, liver, diaphragm, gastrocnemius and kidney tissue. FIG. 3 shows that AON1 achieved sustained dose dependent exon 51 skipping in muscle tissue (quadriceps, Gastrocnemius, heart and diaphragm) with 25 weeks of treatment.

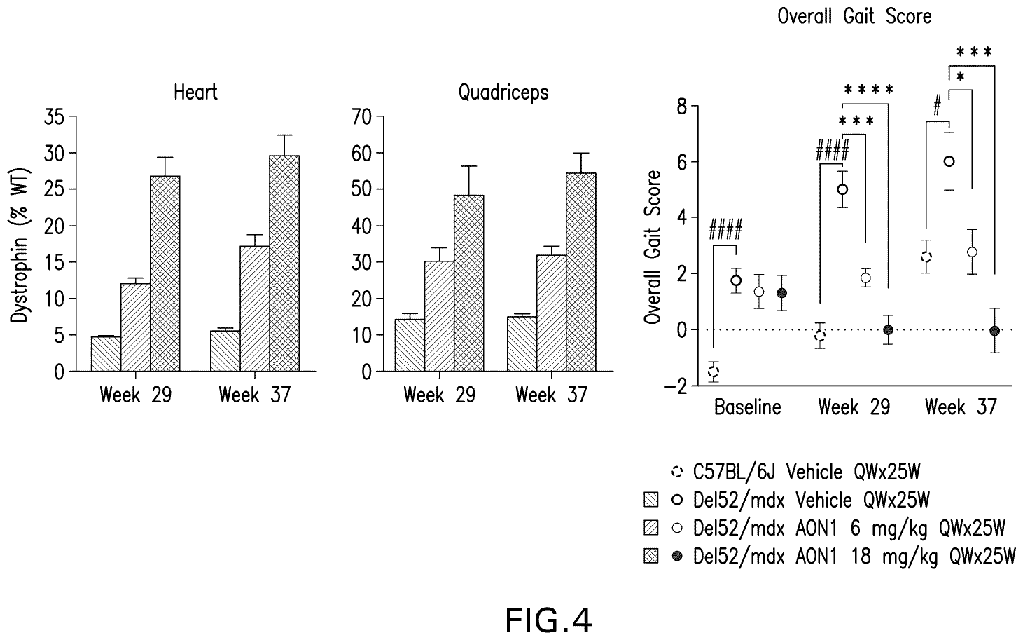

FIG. 4 shows dystrophin wt % in heart and quadreiceps and shows Overall Gait Score for AON1 (Mean±SEM; N=16-18 (Baseline, Week 29) or 10-12 (Week 37). Statistical significances: #p<0.05, ####p<0.0001 compared to C57BL/6J Vehicle; *p<0.05, ***p<0.0005, ****p<0.0001 compared to De152/mdx Vehicle). AON1 improves and sustains dystrophin and gait score with 25 weeks of treatment. Reference for fine motor and kinematic gait testing: NA Datson et al. (2020), NUCLEIC ACID THERAPEUTICS. DOI: 10.1089/nat.2019.0824.

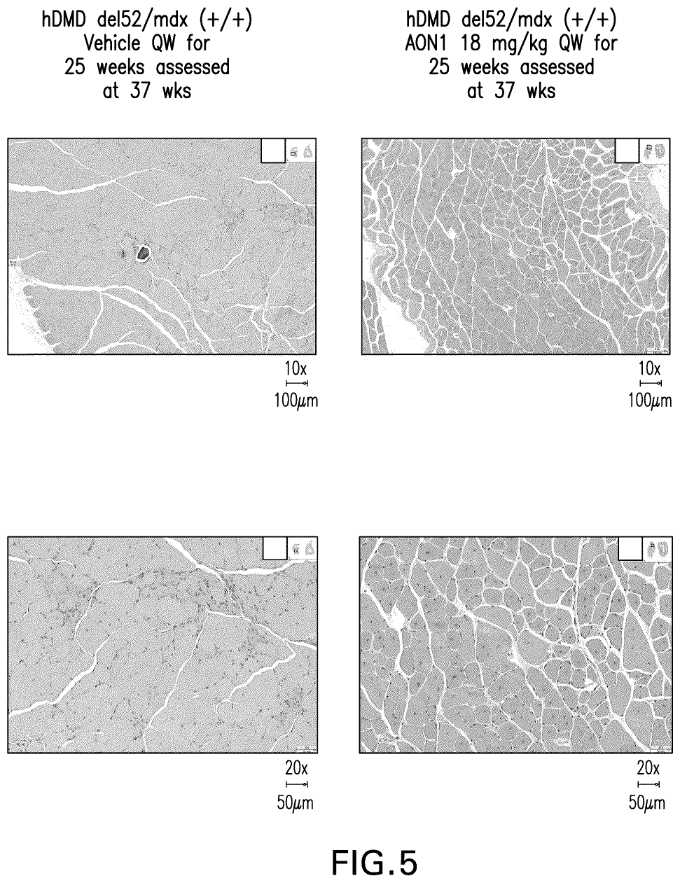

FIG. 5 shows that AON1 treatment attenuates hDMD de152(+/+) model-associated muscle pathology and sustains pharmcological effect for 12 weeks post-25 week dosing. Gastrocnemius muscle from vehicle treated mice showed mild atrophy, minimal necrosis, minimal inflammation (nononuclear cell infiltrate) and minimal fibrosis. In contrast, gastrocnemius muscle from AON1 treated mice showed minimal atrophy without other effects.

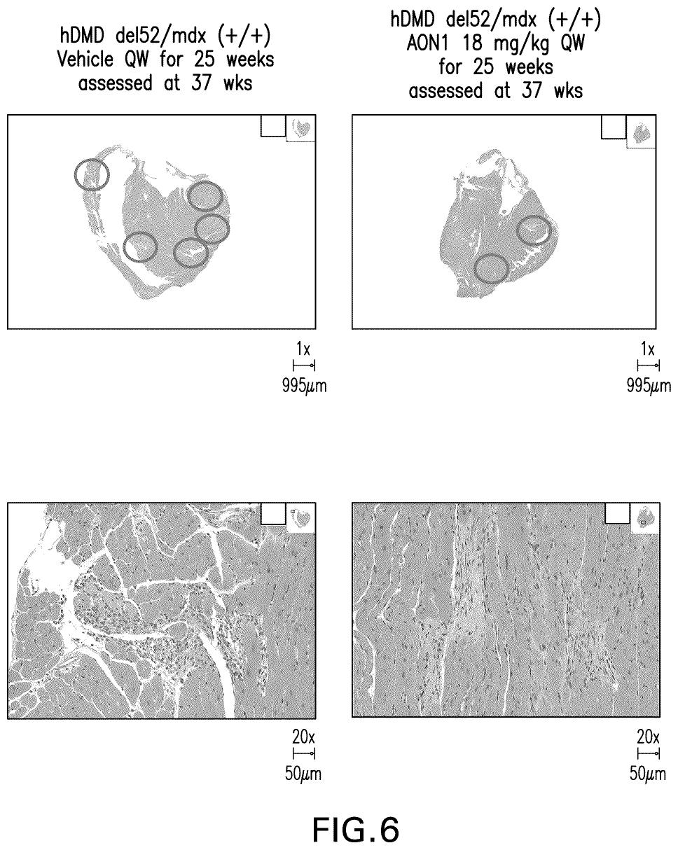

FIG. 6 shows that AON treatment attenuates hDMD de152(+/+) model-associated cardiac pathology and sustains pharmacological effect for 12 weeks. Heart muscle from vehicle treated mice showed minimal atrophy, minimal necrosis, minimal inflammation (mononuclear cell infiltrate) and minimal fibrosis. In contrast, heart muscle from AON1 treated mice showed minimal inflammation (mononuclear cell infiltrate) and minimal fibrosis without other effects.

### Example 2

### Synthesis of AON1

The oligonucleotide, AON1, was synthesized by solid phase organic synthesis. No intermediates were isolated or characterized during the synthetic process. After completion of the synthesis sequence, the oligonucleotide was cleaved from the solid support, deprotected and purified by ultrafiltration and preparative anion exchange (AEX) chromatography. After pooling of HPLC fractions, the oligonucleotide AON1 solution was concentrated and desalted by a second ultrafiltration step. Subsequent lyophilization and homogenization provided the product, AON1, as a solid material which was stored at −20±5° C. prior to shipping.

The synthesis cycle, which was performed at ambient temperature, is schematically shown below (using the first cycle for illustrative purposes). The oligonucleotide chain of AON1 was synthesized on a solid support. After completion of the sequence, the oligonucleotide was cleaved off the resin, purified and the resulting solution was lyophilized to yield the final drug substance, AON1.

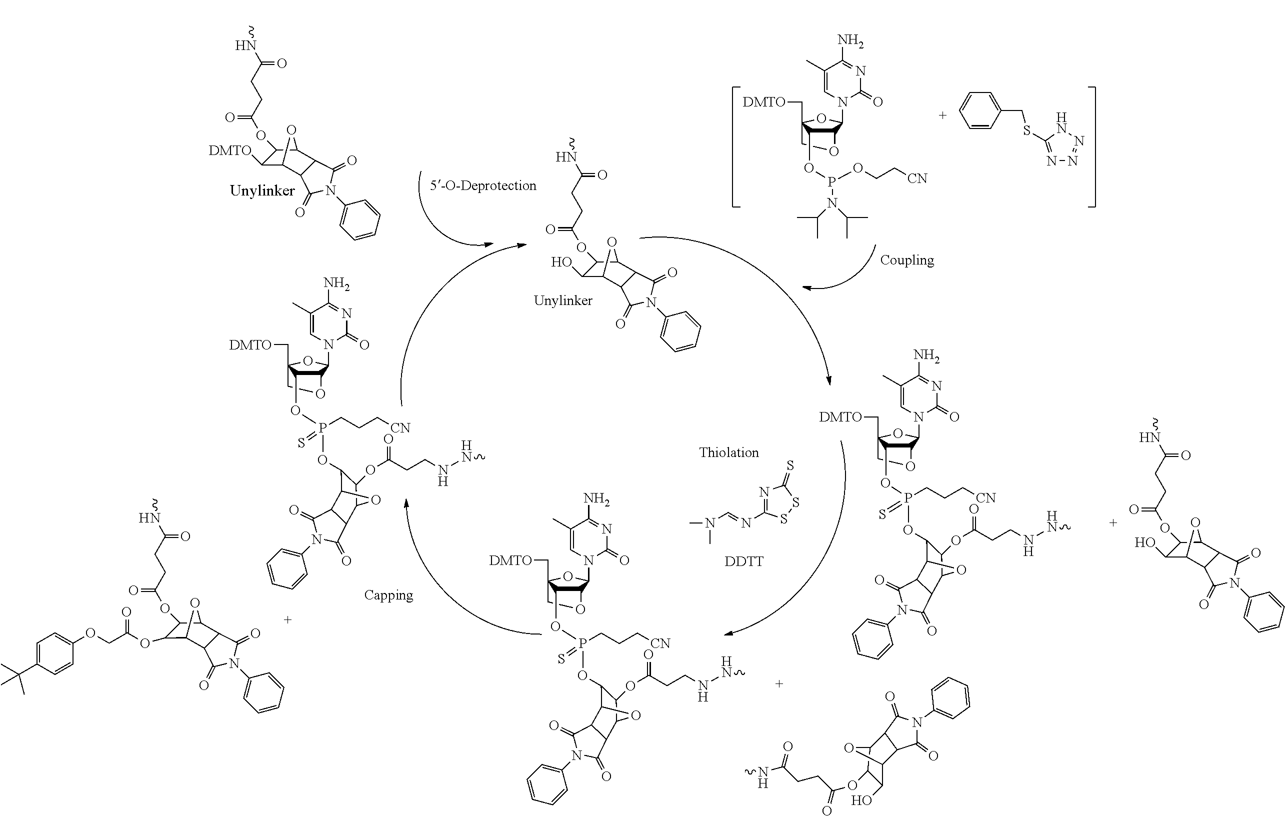

### Stage 1: Solid Phase Synthesis

The oligonucleotide moiety of AON1 was manufactured by solid phase organic synthesis using a platform synthesis. The synthesis was automated and carried out on a synthesizer that was equipped with a closed column reactor, using coupling recycling technology and involved no isolation of intermediates. A solid support with a universal linker was packed into the flowthrough column reactor and the synthesis was initiated. Each synthesis cycle consisted of four chemical steps, which were carried out sequentially, followed by washing, until the full-length oligonucleotide was established.

### Step 1 Deprotection

In the first step, designated as deblocking or deprotection, the solid support was treated with dichloroacetic acid in toluene, to remove the protecting group, dimethoxy trityl (DMT), from the UnyLinker™ on the solid support. In all later cycles of step 1, deblocking involved the removal of the DMT protecting group from the 5′-OH, of each sequentially introduced phorphoramidite or locked amidite, to generate the 5′-DMT-off oligonucleotide.

### Step 2 Activation and Coupling

The second step, designated as “activation & coupling”, was the chain elongation step that added the next nucleotide to the sequence, growing from 3′ to 5′ end. The unprotected alcohol group of either the UnyLinker™ (1st cycle) or the 5′-DMT-off oligonucleotide chain [n] (all following cycles) was treated with a solution of the corresponding phosphoramidite in acetonitrile or dichloromethane/acetonitrile, in the presence of a suitable activator solution, e.g., benzylthiotetrazole in acetonitrile, to build the elongated DMT onto the oligonucleotide chain [n+1]. The final chain elongation step incorporated the TEG group at the 5′ end of AON1 using DMT-TEG-phosphoroamidite without sulfurization.

### Step 3 Oxidation/Sulfurization

Depending on the desired backbone chemistry, the corresponding phosphorothioate or phosphodiester was introduced at this step by sulfurization or oxidation. The DMT-on oligonucleotide chain [n+1] was subjected to a sulfurization agent, e.g., 5-N-(Dimethylamino)methylenelamino-3H-1,2,4-dithiazole-5-thione (DDTT) in pyridine/acetonitrile) or oxidation agent (e.g. iodine in pyridine/water). Only the last cycle involved an oxidation step during the manufacture of AON1, with all other cycles involving sulfurizing reactions.

### Step 4 Capping

The fourth step, designated as “Capping”, finished the synthesis cycle, through adding a mixture of Cap A (acetonitrile/2,6-lutidine/N-methylimidazole) and Cap B (tert-butylphenoxyacetyl acetic anhydride in acetonitrile) to the oligonucleotide on the solid support, in order to prevent any unreacted 5′-OH oligonucleotide chains [n] from further reaction with phosphoramidites in the subsequent coupling cycle. This led to the formation of truncated sequences. After the synthesis cycle for all 18 amidites was completed, the base and backbone protected oligonucleotide remained bound on the solid phase support.

### Stage 2: Backbone Deprotection

The oligonucleotide on the solid phase support was treated with diethylamine in acetonitrile, leading to the removal of the B-cyanoethyl protecting groups on the phosphorothioate/phosphate backbone. The reaction was followed by a washing step using acetonitrile.

### Stage 3: Base Deprotection and Cleavage from the Resin

The column was incubated with aqueous ammonia/ethanol at ambient temperature, followed by washing with aqueous ammonia ethanol. The combined solutions were incubated at elevated temperature. Within this two-step procedure, the crude oligonucleotide was cleaved from the solid support and the nucleobase protecting groups were removed. The resulting solution was filtered and quenched, with the filtrate containing the crude oligonucleotide, dissolved in the deprotection solution.

### Stage 4: Desalting of the Oligonucleotide by Ultrafiltration

The solution of the crude AON1 was concentrated by ultrafiltration using an ultrafiltration membrane. Diafiltration was performed on the resulting concentrate using water for injection. The resulting aqueous AON1 solution was filtered.

### Stage 5: Purification of the Crude Oligonucleotide by Anion Exchange Chromatography

The reaction mixture was diluted in aqueous sodium phosphate/acetonitrile buffer and purified by anion exchange (AEX) chromatography with Source™ 30Q.

Mock pools were generated and analyzed by HPLC-UV/MS. Fractions were selected for pooling and for further down-stream processing, according to the analytical results. The selected fractions were combined to provide a product pool of appropriate purity (>85%) and yield. This final product pool was analyzed again by HPLC-UV/MS.

### Stage 6: Desalting of the Oligonucleotide by Ultrafiltration

The pooled solution of AON1 was concentrated by ultrafiltration using an ultrafiltration membrane. Diafiltration was performed on the resulting concentrate using water for injection. The resulting aqueous AON1 solution was filtered.

### Stage 7: Oligonucleotide as Lyophilized Solids

The concentrated solution from stage 6 was filtered through a 0.2 μm membrane and lastly lyophilized to yield the final drug substance. After lyophilization, the material was equilibrated in the cleanroom environment at minimum for 24 hours, to obtain a stable water content in the final product. The equilibrated drug substance was obtained as a white to off-white to yellow powder (mp: ∫265° C. (DSC decomp); purity 92-93% by ion-pair reversed phase HPLC-UV/MS).

### Example 3

### Process for Manufacturing 50 mg/mL AON1

### Compounding and Optional Bioburden Reduction Filtration of Buffer Solution

The required amount of di-sodium hydrogen phosphate dodecahydrate, sodium dihydrogen phosphate dihydrate, and sodium chloride is weighed in approximately 80% of the total amount of water for injection (WFI) and is stirred in a Grade C environment under laminar air flow (LAF). After the excipients are dissolved, WFI is added to final amount.

If compounding of the buffer solution and compounding of the drug product solution do not occur on the same day the buffer solution is filtered for bioburden reduction through a 0.45/0.2 μm membrane filter unit in Grade C under LAF.

### Equilibration, Compounding, and Bioburden Reduction Filtration of the Solution

The lyophilized AON1 (Example 2) is equilibrated for 12-24 hours at room temperature in a Grade C environment under LAF. After equilibration, the lyophilized AON1 is weighed and dissolved in approximately 50% buffer solution by stirring to avoid clumping. Final AON1 concentration of 50 mg/mL solution is reached by adding a calculated amount of buffer solution. The solution is mixed with 200 to 1000 rpm for 20-30 min by avoiding foaming. The compounded solution is bioburden reduction filtrated through a sterile 0.45/0.2 μm membrane filter unit. Compounding and bioburden reduction filtration is performed in a Grade C environment under LAF.

### Inline Sterile Filtration and Aseptic Filling

The AON1 solution is sterile filtered through two successive in-line filter units using an 0.45/0.2 μm filter and aseptically filled into ready to use sterile vials. The vials are capped and sealed. Filled units are stored at controlled temperature cold storage room.

The AON1 composition is supplied in a single-dose vial as a 3 mL isotonic sterile, preservative free solution for intravenous infusion at a concentration of 50 mg/mL at pH 7.0. The container closure system is a Type 1 glass vial with a fluoropolymer coated bromobutyl rubber stopper and aluminum seal with flip-off cap. The composition of 5.25 L of AON1 solution is provided below:

### Composition of 5.25 L of AON1 Solution

| | | | Concentration |
|---|---|---|---|
| Component | Quantity | | (g/L) |
| AON1 | 262.5 | g | 50 |
| Disodium Hydrogen Phosphate | 27.0 | g | 5.135 |
| Dodecahydrate | | | |
| Sodium Dihydrogen Phosphate Dihydrate | 4.6 | g | 0.883 |
| Sodium Chloride | 24.5 | g | 4.676 |

| | | |
|---|---|---|
| Water for Injection | qs | qs to |
| | | target volume |

qs = quantum sufficit

### Example 4

### AON1 PS Administered for 13 Weeks in Male and Female de152hDMD/mdx Mice with Assessment 2 or 4 Weeks Postdose

A total of 120 homozygous hDMD de152/mdx +/+male and female mice, bred at Charles River, UK, and genotyped by BioLytix, Switzerland, were used for the study. All hDMD de152/mdx mice in this study originated from the re-derived colony. In addition, age-matched C57BL/6J (n=20) mice (Charles River, Germany) were used as wild-type (WT) controls. In setting up the different groups for this study, mice were randomized into groups per sex in such a way that complete litters of mice did not end up in a single testing group and taking the baseline weight into account so that there were no group differences at start of first treatment.

Mice were weighed on each treatment day and the dose was adjusted accordingly. The whole IV injection process took 5-8 minutes per mouse with an actual injection time of 10-60 seconds.

All compounds were formulated at a concentration that ensured equimolar dosing and were administrated at an injection volume of 8 mL/kg. Mice were weighed on each treatment day and the dose adjusted accordingly. For example, a mouse with a 30 g body weight received an IV injection of 240 μL.

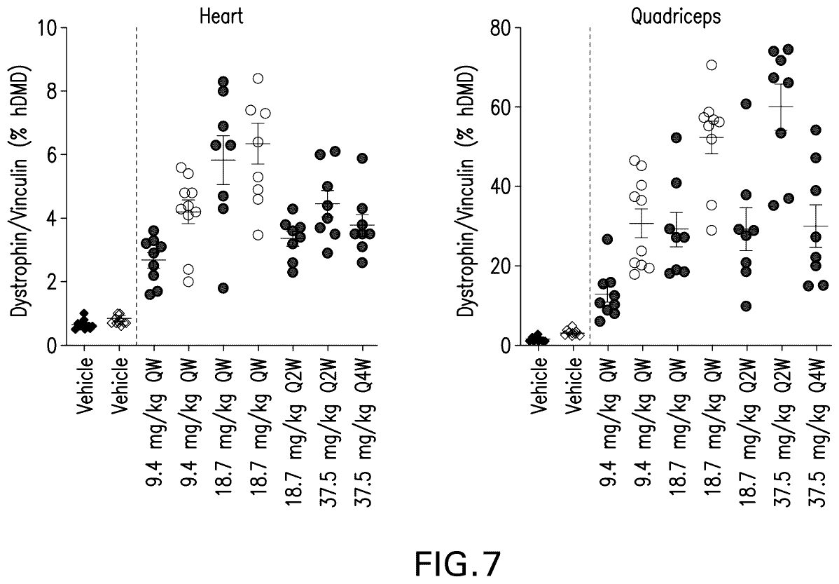

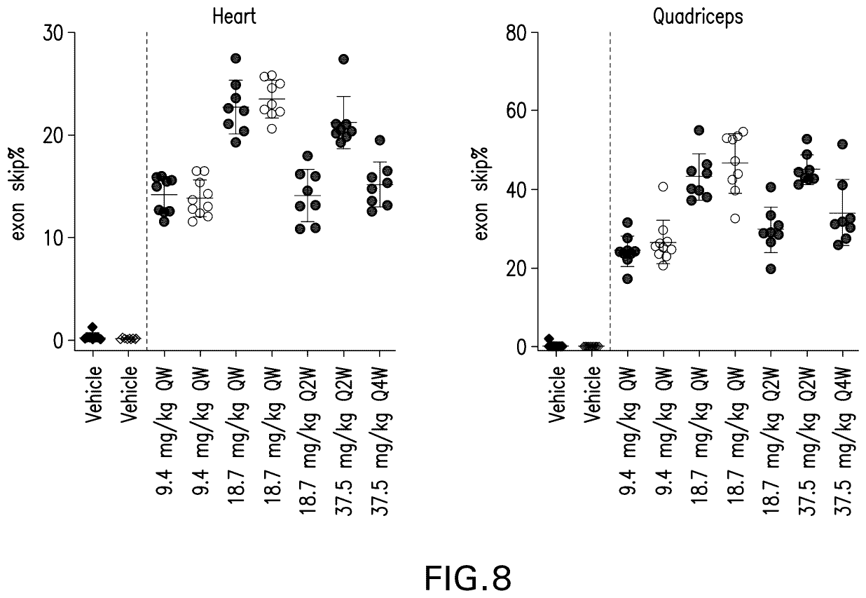

Mice were dosed with AON1 PS for 13 weeks. At the end point, 14 days or 28 days after the last IV injection, the mice were euthanized by deep anesthetization with sodium pentobarbital (60 mg/kg Mebunat, Orion Pharma, Finland). The mice were subjected to cardiac puncture and heart tissue was harvested. The tissue was immediately snap frozen by immersing in isopentane au bain marie in liquid nitrogen, placed in cryovials pre-chilled on dry ice and stored at −80° C. The frozen samples were analyzed for % exon skipping and dystrophin level by qualified methods. Results are shown in FIGS. 7 and 8.

### Example 5

### AON1 Administered for 13 Weeks in Male and Female del52hDMD/mdx Mice With Assessment 4 or 8 Weeks Postdose

All mice received an IV injection in the tail vein once per week (QW) for a study duration maximum of 13 weeks with vehicle or AON1 starting at 7 weeks of age. The whole IV injection process took 5-8 minutes per mouse with an actual injection time of 60 seconds. The compounds were formulated at specified concentrations and administrated at an injection volume of 8 mL/kg. Mice were weighed on each treatment day and the dose adjusted accordingly. For example, a mouse with a 30 g body weight received an IV injection of 240 μL.

At the end of in-life, 4 or 8 weeks (Days 113 and 141, respectively) after the last IV injection on Day 85, following blood collection via cardiac puncture, mice were transcardially perfused with PBS in order to remove blood from the tissues. The following tissues were collected and snap frozen:

Skeletal muscles: gastrocnemius (R), quadriceps (L+R); collect both sides into one vial so quadriceps tissue has one sample.

One half of the heart (dissected so that both atrium and ventricle are present in each half), the entire diaphragm, liver (2×⅓rd of the total liver), kidney (R).

All tissues were immediately snap frozen by immersing in isopentane au bain marie in liquid nitrogen, placed in cryovials pre-chilled on dry ice and stored at −80° C. until sectioning.

Exon skipping analysis was performed in gastrocnemius, quadriceps, heart, and diaphragm tissues from all groups. The analytical work was conducted using a qualified reverse transcriptase digital droplet polymerase chain reaction (RT-ddPCR) analytical method. hDMD exon 51 skip percent was calculated by the following formula:

(Human Skip Exon 51 copies/20 μL reaction)/(Human Skip Exon 51 copies/20 μL reaction+Human Nonskip Exon 51 copies/20 μL reaction)*100

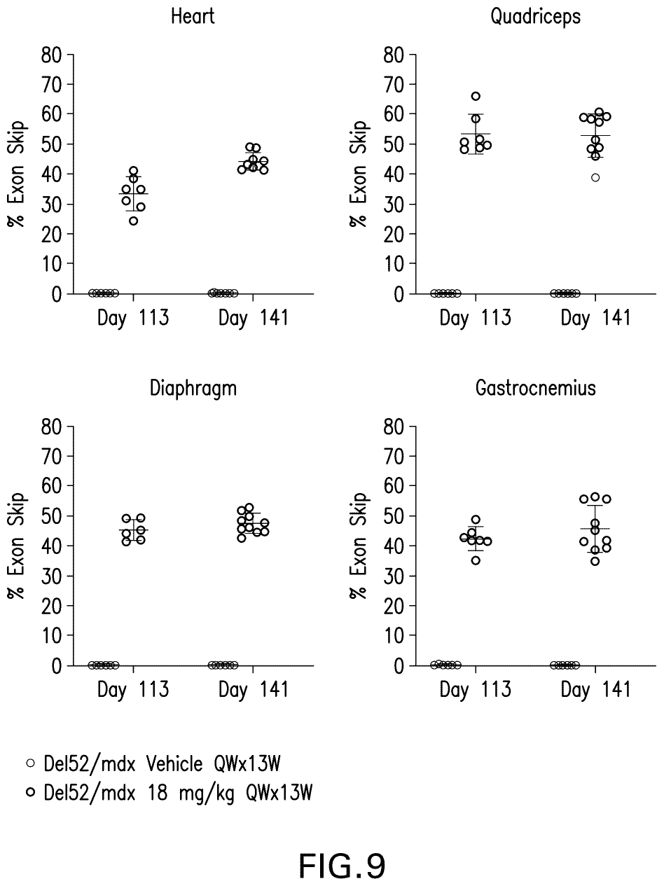

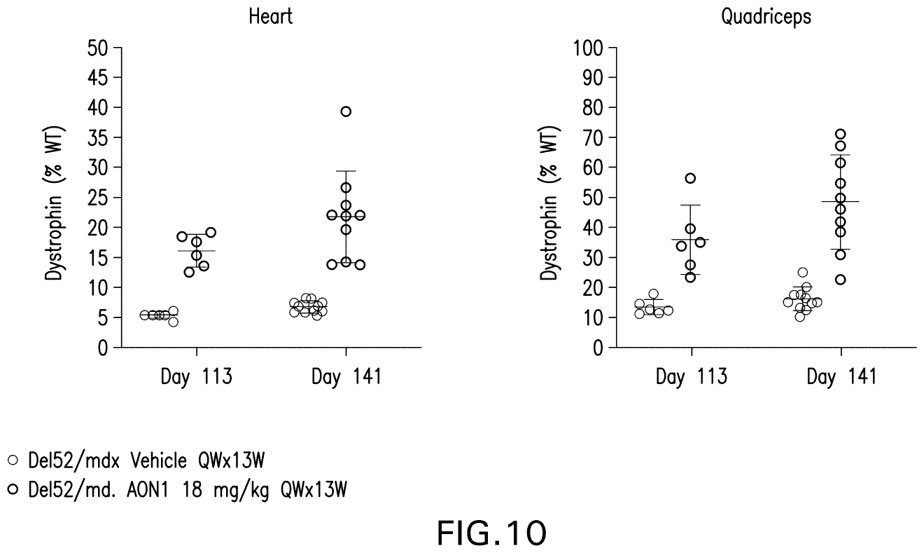

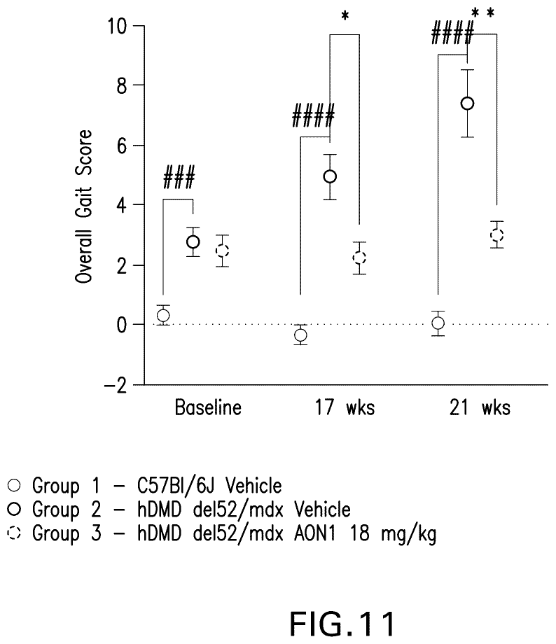

The lower limit of quantitation (LLOQ) for skip and non-skip transcripts was based on a threshold approach. Results are shown in FIG. 9. Dystrophin levels in heart and quadriceps are shown in FIG. 10. Overall Gait Score is shown in FIG. 11.

### Example 6

### A Study of the Effect of AON1 PS and AON2 on Complement Activation in Human and Xynomolgus Monkey Serum was Conducted

The dose vehicle for test article administration was aqueous phosphate buffer (pH 6.9-7.1) with 0.7% NaCl, which was prepared and used as a baseline control. The appropriate amount of test article (AON1 PS or AON2) was weighed and dissolved into a visually-clear solution at 10× dose concentration using stirring/vortex mixing. No correction factor was applied to the weight as measured. Formulations were stored refrigerated (4° C.) before and after use. Before use, all test article formulations were warmed to room temperature on the on benchtop for at least 30 minutes. Test articles were considered stable at 4° C. storage for one week following preparation. Actual weights and dilutions were recorded in the study data. No dose analysis occurred for this study; residual formulations were discarded.

### Test System

Test System 1: Individual serum from n=3 normal male human volunteers (>18 years of age).

Test System 2. Individual serum from n=3 normal male cynomolgus monkeys (>3 years of age).

### Route of Administration

This was an in vitro study, and the test articles were exposed to the test system in vitro. The test articles were added to the test system at a ratio of one part to nine parts (1:9, v/v). This ratio maintained appropriate concentration of the test system so there was sufficient concentration of the complement control protein.

### Dose Regimen

The mix was incubated at 37° C.±2° C. for 30±2 minutes and stored at −80° C. until analyte testing.

### Objective and Study Protocol

The purpose of this study was to evaluate the complement split product activation profiles for two antisense oligonucleotides (AON1 PS and AON2) following incubation in normal male human and cynomolgus monkey serum.

### Experimental Design

The testing was performed by mixing 270 μL of test system with 30 μL of prepared test article in a 1.5 mL polypropylene microcentrifuge snap cap tube. Once all the mixtures were prepared, they were transferred to 37° C.±2° C. water bath and incubated with for 30±2 minutes. After the mixtures were incubated, all samples were aliquoted and frozen at −80° C. or below.

### Bioanalytical Analysis of Complement Activation

Bb Split Product by ELISA: Bb is created upon cleavage of Factor B by activation of the alternative pathway of complement. The level of Bb produced during in vitro exposure to the test article can act as a measure of the level of alternative pathway activation. The Bb ELISA is run with duplicate wells for which an average value is reported. The level of Bb was assessed initially at two dilutions to verify the assay was not saturated by the higher activation samples.

C3a Split Product by ELISA: C3a is created upon cleavage of C3 at the central point of complement. As such, C3a cleavage can result following activation of any of the three pathways of complement. The level of C3a is also important as it is an anaphylatoxin. The level of C3a produced during in vitro exposure to test article can act as a measure of the level of complement activation strong enough to reach this central point, as well as act as an indication of the likelihood of direct proinflammatory outcome of this activation. The C3a ELISA is run with duplicate wells for which an average value is reported. The level of C3a was assessed initially at two dilutions to verify the assay was not saturated by the higher activation samples.

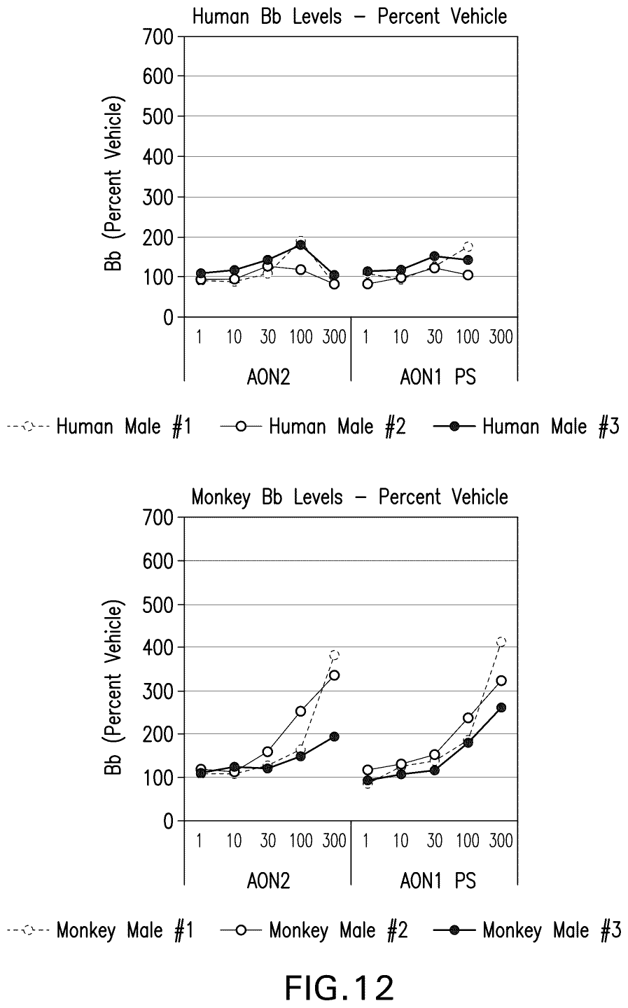

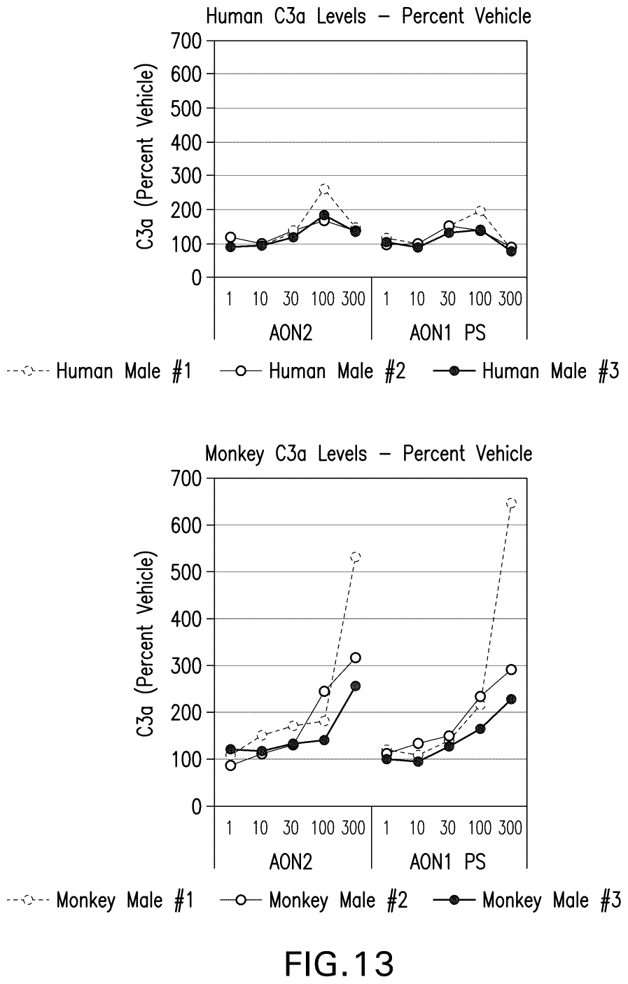

Results are shown in FIGS. 12 and 13.

### Example 7

### Juvenile Mouse 26-Week Toxicity and Bioanalysis Study

In a 26-week juvenile toxicity and toxicokinetic (TK) study with 13 weeks recovery, male CD-1 mice (20-22/group (main toxicity study); 6 (vehicle; vehicle control buffer diluted with sterile saline), 25-31/group (TK); 21-27/group (recovery)) were dosed via bolus IV injection with 0 (vehicle control buffer), 6, 12, or 18 mg/kg AON1 once every 4 days from PND 21 through PND 203. Blood was collected from AON1-treated groups on PND 21 and 203 at predose (PND 203 only), 0.5, 1, 3, 8, and 24 hours postdose for bioanalyses. The following parameters were assessed: body weight, food consumption, developmental landmarks (balanopreputial separation), neurobehavioral assessments (locomotor activity at ≈PND 187 and 281; auditory startle at ≈PND 190 and 284; Morris water maze at ≈PND 193 and 287), ophthalmic examinations, clinical pathology (clinical chemistry, hematology, and coagulation), gross necropsy, organ weights, and histopathology.

Based on the lack of adverse findings, the no observed adverse effect level (NOAEL) for toxicity was 18 mg/kg/dose. On PND 21, this dose level corresponded to maximum observed concentration (Cₘₐₓ) and area under the concentration-time curve from 0 to 24 hours postdose (AUC₀₋₂₄) values of 5280 nmol/L and 11,700 h* nmol/L, respectively. On PND 203, this dose level corresponded to Cmax and AUCo-24 values of 5860 nmol/L and 14,600 h* nmol/L, respectively.

### Example 8

### NHP 39-Week Toxicity and Bioanalysis Study

Male cynomolgus monkeys (3/group (main toxicity study); 2/group for 0, 6, 12, and 18 mg/kg QW (13-week recovery)) were dosed via IV infusion (60 minutes) with 0 (vehicle: NaCl Injection, USP), 6, 12, or 18 mg/kg QW AON1 for 39 weeks. Blood was collected for bioanalyses/TK and for complement activation analysis on different study days/timepoints. The following parameters were evaluated: mortality, clinical signs, body weight, food consumption, AON1 levels in plasma and tissues (gastrocnemius, heart, biceps femoris, liver, and kidney), percent exon skipping in tissues (gastrocnemius, heart, biceps femoris, liver, and kidney), clinical pathology (clinical chemistry, hematology, urinalysis, urine chemistry, and urine biomarkers), complement activation indicators (Bb, C3a, and sC5b-9), ophthalmology, ECG, organ weights, and histopathology.

Mean concentrations of AON1 in tissues were quantifiable in all AON1-treated groups at the scheduled terminal euthanasia on Day 274 and at unscheduled euthanasia. The mean concentrations generally increased with an increased in dose from 6 to 12 mg/kg/dose. The rank order of mean concentrations of AON1 on Day 274 of the dosing phase from 6 to 12 mg/kg/dose was the liver, kidney, heart, gastrocnemius, and biceps femoris.

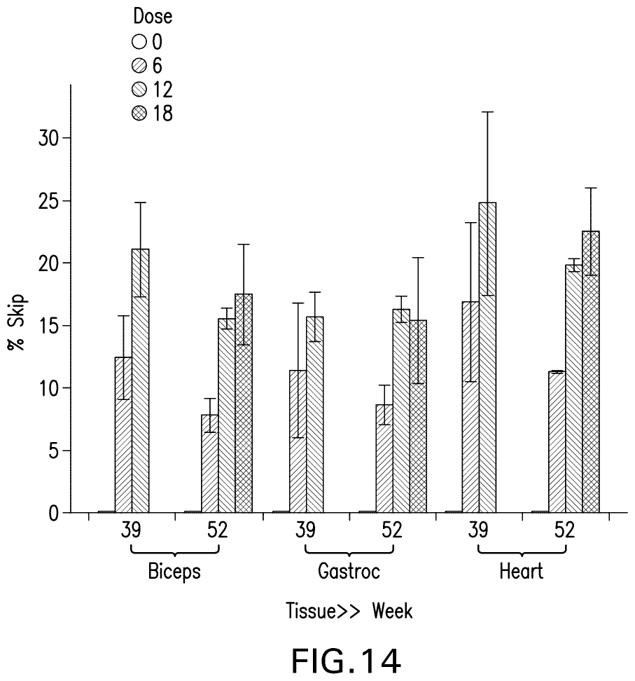

Exon skipping in tissues was measured by RT-ddPCR. The method quantified the number of non-skipped DMD (dystrophin) transcripts, and the number of exon 51-skipped DMD transcripts; and the percent exon skipping (% skip) was calculated. The percent exon 51 skip varied from 4.5% to 40.8% and was higher in the high dose groups (12 and 18 mg/kg/QW) than in the low dose group (6 mg/kg/QW). See also FIG. 14.

No AON1-related body weight changes; ophthalmic findings; veterinary observations; abnormal ECG waveforms or arrhythmias; or effects on PR interval, QRS duration, QT or corrected QT (QTc) interval, or heart rate were noted. No AON1-related clinical pathology (hematology and coagulation) effects were observed for animals administered up to 18 mg/kg/dose. No AON1-related macroscopic observations or organ weight effects were noted at the early terminal sacrifice or scheduled terminal sacrifice for males administered up to 18 mg/kg/dose.

One male each administered 12 or 18 mg/kg/dose were sacrificed in a AON1-related moribund condition on Days 194 or 211 of the dosing phase, respectively. Clinical and veterinary observations were similar in both animals, and included decreased activity, thin appearance, and pitting or subcutaneous edema of the lower trunk and/or limbs. Clinical pathology findings indicated inflammation, azotemia, hypoalbuminemia, and elevated urinary protein (in one animal where it was measured). The cause of moribundity was attributed to a glomerular kidney injury and multisystemic vascular inflammation. AON1-related clinical pathology effects in unscheduled sacrifice animals supported an inflammatory response, fluid deficit, possible gastrointestinal loss, excitement/physiologic response, and/or moderately decreased platelet count, which lacked a clear mechanism. Microscopic findings for unscheduled sacrifice animals included kidney tubular degeneration/regeneration, vascular degeneration, hypertrophy, and inflammation of the vessel wall and/or perivascular tissue, which may have resulted in interstitial edema, thrombosis (lung), and/or a localized degenerative change in the affected tissue (heart). Edema may also have been associated with markedly lower serum protein, which was due to glomerular changes. These findings and their sequalae were consistent with a complement-mediated etiology, to which monkeys are prone in the context of AON therapy (Frazier Toxicol Pathol. 2015 January;43(1):78-89). The reason for increased sensitivity to AONs in NI-IPs has been linked to the genetics of the Factor H component in cynomolgus monkeys that is understood to increase the binding of Factor H to AONs. The reduction in the amount of free Factor H required to regulate complement activation removes the key regulator of the alternative pathway.

Low level and transient complement activation involving alternative and terminal pathways of complement was observed in all dose groups, including the control group. The increases in complement activation generally decreased by 72 hours post dose to near predose levels in most animals, with a trend of increasing levels of activation over the course of the study. The two early terminal sacrificed animals had >200% increase in Bb levels on Day 113 and Day 204, respectively, compared to predose levels. While the complement activation profile for these two animals is consistent with complement being involved with the kidney damage observed, other animals also exhibited increases in the measured complement fragments. The differences among animals could be due to individual differences in the animal's ability to control complement activation once initiated as there are a number of complement regulators. The mechanism of complement activation through nonspecific interaction between the AON and Factor H protein was noted to have very limited clinical relevance because human complement activation occurs at much higher AON plasma concentrations (Henry Int. Immunopharmacol. 2002;2:1657-66; Henry Antisense Drug Technology: Principles, Strategies, and Applications, 2nd ed. CRC Press: Carlsbad, CA; 2008:327-63; Shen J Pharmacol Exp Ther. 2014 December;351(3):709-17).

AON1-related clinical pathology effects were observed in scheduled sacrifice animals administered ≥6 mg/kg/dose. Mildly increased alanine aminotransferase (ALT) and alkaline phosphatase (ALP) activities on Days 176 (except ALT), 204, 211, 216, and 218 of the dosing phase in animals administered 18 mg/kg/dose suggested a hepatic disturbance. Effects that supported an inflammatory response included mildly decreased albumin concentration and increased globulin concentration (which resulted in a decreased albumin:globulin ratio) on Days 176, 204, 211, 216, and 218 of the dosing phase in animals administered 18 mg/kg/dose and mildly increased haptoglobin concentration on Days 211, 216, and 218 of the dosing phase in animals administered 18 mg/kg/dose. In addition, albumin was lost via the kidney, which was supported by the mildly increased urine protein:urine creatinine ratio and an increased incidence of protein in the urine on Days 211, 216, and 218 of the dosing phase in animals administered 18 mg/kg/dose. Mildly to moderately increased microalbumin:creatinine ratio on Days 211, 216, 218, 225, 239, 253, 260, 267, and 274 of the dosing phase in animals administered ≥6 mg/kg/dose also supported albumin loss via the kidney. Mildly increased clusterin:creatinine ratios on Days 211, 216, and 218 of the dosing phase in animals administered 18 mg/kg/dose supported a kidney injury. An additional effect was non-dose-dependent, mildly decreased platelet count on Days 176, 204, 211, 216, 218, 225, 239, 253, 260, 267, and 274 of the dosing phase in animals administered ≥6 mg/kg/dose, which lacked a clear mechanism. Decreases in platelet counts over time leading to moderate/marked thrombocytopenia was observed in 2 animals in the 18 mg/kg QW group, although not associated with any anatomic pathology findings. No AON1-related coagulation effects were observed in scheduled sacrifice animals.

Microscopic findings in two early terminal sacrifice animals administered 18 mg/kg/dose on Day 218 of the dosing phase and scheduled terminal sacrifice animals involved minimal degeneration of the renal tubular epithelium in one animal and minimal mixed cell inflammation in the liver of another animal, which correlated with increased liver enzyme values. The findings were attributed to an accumulation of AON1 within the renal tubular epithelium or Kupffer cells, respectively. Additional AON1-related findings, common in moribund and early sacrifice animals, included slight or moderate mononuclear cell infiltrates in the heart valves and a trend toward an increased incidence of mononuclear infiltrates in several tissues. These findings likely reflected the presence of systemic inflammation and/or complement activation associated with dosing and, with the exception of valvular changes, were considered an exacerbation of commonly observed incidental findings in monkeys. In all animals, observations related to an accumulation of AON1 included basophilic granules in the renal tubular epithelium and Kupffer cells (generally in a dose-related manner) and the presence of macrophages characterized by an altered, foamy and/or slightly basophilic cytoplasm in multiple tissues but most notably in the lymph nodes. The accumulation appeared not to be associated with damage to surrounding tissues of animals administered ≥12 mg/kg/dose, and macrophage alterations appeared not to be associated with damage at any dose. At the recovery euthanasia, microscopic findings trended towards reversibility, as demonstrated by minimal basophilic granules in the kidney, minimal pigment in the Kupffer cells of the liver, and minimal to moderate vacuolated/pigmented macrophage infiltrates in the mandibular and/or mesenteric lymph nodes of animals administered ≥6 mg/kg/dose. Additionally, minimal vacuolated/pigmented macrophage infiltrates were noted in the gastrointestinal tract of one animal each administered 12 or 18 mg/kg/dose and in the urinary bladder (no transitional cell degeneration/vacuolation was noted in this monkey study) and testis of one animal administered 18 mg/kg/dose.

Based on the clinical pathology conditions and the severity of anatomic pathology findings in terms of the overall impact on the health and well-being of males administered AON1, the no observed adverse effect level (NOAEL) was considered 6 mg/kg/dose, which corresponded to a mean maximum observed concentration (Cₘₐₓ) of 17,000 nmol/L and area under the concentration-time curve (AUC₀₋₂₅) of 36,500 h*nmol/L, on Day 260 of the dosing phase.

### Example 9

### First In Human Dose Selection

The proposed doses and duration of the clinical trial (Example 10) were selected to ensure safety as well as a prospect of direct benefit to each pediatric participant enrolled in the trial.

The first in human clinical study (Example 10) includes the sequential evaluation of up to 6 dose levels of AON1: 0.6 mg/kg, 1.5 mg/kg, 3 mg/kg, 6 mg/kg, 9 mg/kg, and 12 mg/kg administered IV.

The starting dose of the clinical study (Example 10) was determined using the NOAEL of AON1 from a GLP 13-week repeat-dose toxicity study in male cynomolgus monkeys (18 mg/kg/week) (Example 13). The Human Equivalent Dose (HED) of the monkey NOAEL of 18 mg/kg QW is 5.8 mg/kg QW, based on a body surface area scaling approach. Thus, the selected starting dose of 0.6 mg/kg provides a 10-fold safety margin based on the monkey, which is considered to be a more relevant species for hazard identification, as monkeys are more sensitive to complement activation and the consequent inflammatory response, which is regarded as one of the class effects of antisense oligonucleotides (the latter being the primary source of nonclinical toxicity in monkeys) (Shen J Pharmacol Exp Ther. 2014 Dec;351(3):709-17). It is widely accepted that the use of monkeys will maximize the likelihood of identifying any adverse responses that are quantitatively and qualitatively similar to those which may be expected in humans (Farman Toxicol Pathol. 2003;31:119-122).

Given the low accumulation expected due to the short plasma half-life of AON1 compared to a once weekly dosing interval, increasing the dose level every 2 weeks for Cohort 1A (Part 1) is considered appropriate to minimize the duration of exposure for an individual participant at a sub-therapeutic dose level. A 2- to 2.5-fold dose increment permits a safe yet efficient escalation of individual participants through doses which nonclinical data indicate are likely to be safe. The toxicology findings observed in the 39-week study are associated with chronic dosing and are not expected with the initial single ascending doses in the clinical trial (Example 10).

Plasma exposure for phosphorothioate oligonucleotides have been shown previously to be similar between NHPs and DMD patients for the same mg/kg doses (Bosgra Nucleic Acid Ther. 2019;29(6):305-322); thus, use of body surface area scaling provides a more conservative approach for starting dose selection for single ascending dose portion of the clinical trial than body weight scaling. For the repeat dose phase of the clinical trial, direct body weight-based (mg/kg) extrapolation from NHPs to humans is applied and sequential evaluation of 6, 9, and 12 mg/kg QW is proposed.

The starting dose of 6 mg/kg QW for the repeat-dose phase is selected to enable each pediatric participant to have a prospect of direct benefit, as this is a pharmacologically active dose at which dystrophin expression is expected to be >10% of normal at steady state. The DMC will review the safety data for the dosing cohort through Week 4 and make a recommendation on dose escalation.

The maximum dose of 12 mg/kg/week, which has been selected to maximize the potential efficacy of treatment with BMN 351 with a manageable safety profile, is justified and informed by the chronic toxicology studies:
Overall, the principal toxicity signals in the chronic toxicology studies (the 39-week monkey study (Example 8), 26-week mouse study (Example 11), and juvenile mouse study (Example 7)) are consistent with well-known class effects of phosphorothioate oligonucelotides that are all clinically monitorable.
Dose limiting toxicities were observed in the 39-week monkey study, where the NOAEL was determined to be 6 mg/kg/week. Dosing above the NOAEL identified in the 39-week monkey study (6 mg/kg/week) is considered appropriate as the dose limiting toxicities at ≥12 mg/kg QW appear consistent with complement-mediated effects to which NHPs are known to have exaggerated sensitivity compared to other preclinical species and humans (Barbour Nephrol Dial Transplant. 2013 July;28(7):1685-93; Shen J Pharmacol Exp Ther. 2014 December; 351(3):709-17). As such, complement-mediated effects should be considered hazard identifying rather than providing a margin of clinical safety. Decreases in platelet counts over time leading to moderate/marked thrombocytopenia was observed in 2 animals in the 18 mg/kg QW group in the 39-week monkey study, although not associated with any anatomic pathology findings. Platelet reductions are monitorable in the clinic.

The liver, kidney, hematologic (thrombocytopenia), coagulation system, and vascular system (inflammatory changes) were identified as the target organs for human toxicity based on nonclinical studies.

Risk monitoring and mitigation activities will be conducted through frequent clinical and laboratory monitoring including the use of organ specific toxicity biomarkers, clearly outlined DLT criteria, and study stopping criteria. Safety data will be evaluated before the Data Monitoring Committee (DMC) recommends whether to dose escalate, dose de-escalate, open new cohorts, or expand cohorts.

### Example 10

### A Phase ½ Dose Escalation Study to Assess the Safety, Tolerability, Pharmacokinetics, and Pharmacodynamics of AON1 in Participants with Duchenne Muscular Dystrophy

This study will investigate the safety and tolerability of AON1 at 6 escalating doses via intravenous (IV) infusion. Given the progressive nature of Duchenne muscular dystrophy (DMD), this is an open-label study and all participants will receive AON1. The proposed doses and trial duration are selected to ensure safety as well as a prospect of direct benefit to each pediatric participant enrolled in the trial. The drug product to be used is described in Example 3.

The no observed adverse effect level (NOAEL) from a pivotal 13-week GLP tox study in monkeys was determined to be 18 mg/kg QW; therefore, the human equivalent dose (HED) is 5.8 mg/kg QW, based on a body surface area scaling approach. Thus, the selected starting dose of 0.6 mg/kg provides a 10-fold safety margin based on the monkey, which is considered to be more relevant for hazard identification, as monkeys are more sensitive to complement activation and the consequent inflammatory response, one of the class effects of antisense oligonucleotides (Shen L, Frazer-Abel A, Reynolds P R, et al. Mechanistic understanding for the greater sensitivity of monkeys to antisense oligonucleotide-mediated complement activation compared with humans. J Pharmacol Exp Ther. 2014 December;351(3):709-17). The low (2- to 2.5-fold) dose titration will further increase the likelihood of early detection of any potential dose-dependent safety signals and thus allow for earlier intervention.

Dosing of participants at each of the 6 dose levels (0.6 mg/kg, 1.5 mg/kg, 3 mg/kg, 6 mg/kg, 12 mg/kg, and 18 mg/kg) will be staggered with intervals based on prior safety experience with this class of molecule. The Data Monitoring Committee (DMC) will evaluate available safety data before recommending whether to dose escalate, dose de-escalate, open new cohorts, or expand cohorts. This design includes careful monitoring of participant safety, including the acute safety events that are known class effects of antisense oligonucleotides (AONs) prior to dose escalation and, if necessary, the implementation of individual and study stopping rules.

The study is being conducted to demonstrate proof of concept via dystrophin expression in conjunction with safety, tolerability, and pharmacokinetics (PK) to evaluate dose response and provide insights into dose selection for further clinical development. Studies have shown that the higher the amount of dystrophin in the muscle, the less severe the disease phenotype (de Feraudy et al. Ann Neurol. 2021;89(2):280-292); hence, increased dystrophin expression is a meaningful outcome for this study.

Plasma PK and muscle distribution will be assessed to investigate the relationship between dose, plasma and muscle exposure, exon skipping, and dystrophin expression.

A readout of AON1 distribution, exon skipping, and dystrophin levels in muscle will occur after all participants complete 12 (Cohort 1A, Part 2) or 24 weeks (Cohorts 1B, 2, and 3) of treatment at the 6 mg/kg, 9 mg/kg, and 12 mg/kg dose levels to determine the optimal tolerated dose(s) to carry into future clinical development.

Once this is completed, participants may enroll into a long term extension (LTE) study with a planned duration of at least 1 year. The rationale for the extension phase is to assess the safety, tolerability, PK, and pharmacodynamic (PD) effects of AON1 in participants with DMD over a longer period of time, as well as to assess within participant functional change longitudinally and potentially compare to a matched external control group. Given the natural history of the disease, an associated extension study is a necessary component of this study design.

| | |
|---|---|
| Objectives | Endpoints |

Primary

| | |
|---|---|
| To assess the safety and tolerability | Incidence of adverse |
| of AON1 at different dose levels in | events/SAEs/AESI |
| participants with DMD | Physical examination |
| | Safety laboratory test parameters |
| | ECG parameters |
| | Echocardiography |

Secondary

| | |
|---|---|
| To evaluate the plasma and urine | AON1 plasma PK |
| pharmacokinetics and muscle | AUC₀₋ₜ, AUC_{0-inf} |
| distribution of AON1 | Cₘₐₓ, C_{trough} |
| | Tₘₐₓ, CL, Vₛₛ, t_{1/2} |
| | AON1 urine PK |
| | Ae_{0-24 hr}, CL_{R} |
| | AON1 concentration in muscle at |
| | Week 13 (Cohort 1A) or Week 25 |
| | (Cohorts 1B, 2, and 3) |

Exploratory

| | |
|---|---|
| To evaluate the effect of AON1 on | Dystrophin expression in muscle |
| exon 51 skipping and dystrophin | biopsy (change from baseline at Week |
| expression in muscle in participants | 13 (Cohort 1A) or Week 25 (Cohorts |
| with DMD | 1B, 2, and 3)) |
| | Exon skipping efficiency (RT-ddPCR |
| | on dystrophin messenger ribonucleic |
| | acid (mRNA) from muscle biopsy) |
| | (change from baseline at Week 13 |
| | (Cohort 1A) or Week 25 (Cohorts 1B, |
| | 2, and 3)) |
| To evaluate the immune response to | Anti-AON1 antibodies |
| AON1 | Anti-dystrophin antibodies |
| To evaluate the effect of AON1 on | NSAA (change from baseline at Week 25) |
| physical function | 6MWT (change from baseline at Week 25) |
| To evaluate the risk of suicidality | Child Behavior Checklist (CBCL) |
| in participants receiving AON1 | |

6MWT, 6 minute walk test; AE, adverse event; AESI, adverse event of special interest; AUCo-t, area under the concentration-time curve from Time 0 to the last timepoint; AUC_{0-inf}, area under the concentration-time curve from Time 0 to infinity; CBCL, Child Behavior Checklist; CL, clearance; Cₘₐₓ, maximum concentration; C_{trough}, trough plasma concentration; DMD, Duchenne muscular dystrophy; ECG, electrocardiogram; mRNA, messenger ribonucleic acid; NSAA, NorthStar Ambulatory Assessment; PK, pharmacokinetics; RT-ddPCR, reverse transcriptase-droplet digital polymerase chain reaction; SAE, serious adverse event; t112, half-life; Tₘₐₓ, time to maximum concentration; Vₛₛ, volume of distribution at steady state

This study is a dose escalation study to assess the safety, tolerability, pharmacokinetics, and pharmacodynamics of 6 escalating doses of IV AON1 in participants with DMD who are amenable to exon 51 skipping. This is a Phase ½, open-label study; no randomization is planned. Data will be compared against individual participants' baseline data. To be eligible for the study, potential participants must meet all eligibility requirements described in the protocol, including the following selected key inclusion criteria:
Is male and age 4 through 10 years at Screening
Clinical diagnosis of Duchenne muscular dystrophy resulting from a documented dystrophin mutation in the DMD gene amenable to exon 51 skipping as reviewed by a central genetic counselor
Ambulatory at Screening, defined as able to walk independently without assistive devices and complete the timed 10 meter walk/run test in 8 seconds or less
Not currently daytime ventilator dependent and not expected to need daytime mechanical or noninvasive ventilation within the next year
Currently receiving treatment with oral corticosteroids, on a stable dose for at least 12 weeks prior to Baseline, and must remain on a consistent dose/dose regimen throughout the study except for modifications to accommodate changes in weight
Normal kidney function based on prespecified laboratory values

Selected key exclusion criteria include the following:
For children 7 years of age or older, forced expiratory volume (FEV₁)<60% of predicted
Current or history of liver or renal disease
Left ventricular ejection fraction (LVEF) <55% based on an echocardiogram (ECHO) performed within 3 months prior to Screening or at the Screening visit
Mean QT interval corrected with Fridericia's method (QTcF) ≥450 msec on the Screening electrocardiogram (ECG) conducted in triplicate
Platelet count of <150×10⁹/L at Screening
Treatment with any exon 51 skipping therapy within 12 weeks prior to Baseline (Day 1) or with any gene therapy for the treatment of DMD at any time

Eligible participants will be assigned to 1 of 3 cohorts: Cohort 1 (n=6), Cohort 2 (n=3), or Cohort 3 (n=6). Cohort 1 participants are further divided into Cohort 1A (n=3) and Cohort 1B (n=3). In Cohort 1A (Part 1), participants will receive single ascending doses of AON1 at 0.6 mg/kg, 1.5 mg/kg, 3 mg/kg and 6 mg/kg, followed at each dose level by a 1 week dosing holiday when safety data is assessed prior to escalation to the next higher dose or, in the case of the 6 mg/kg dose, escalation to chronic dosing. In Cohort 1A (Part 2), participants receive weekly doses of AON1 at 6 mg/kg, the dose expected to result in approximately 10% dystrophin expression at steady state based on nonclinical data. Cohort 1B participants will receive 6 mg/kg QW, Cohort 2 participants (n=6) will receive 9 mg/kg QW, and Cohort 3 participants (n=6) will receive 12 mg/kg QW. The detailed dose escalation procedures are described below.

A DMC will monitor safety and make dose escalation recommendations. Dose-limiting toxicity (DLT) criteria are applicable to the period through assessments 1 week after the single doses at 0.6 mg/kg, 1.5 mg/kg, 3 mg/kg, and 6 mg/kg (Cohort 1A (Part 1) only). For Cohorts 1A (Part 2), 1B, 2, and 3, DLT criteria are applicable through the Week 4 evaluation of the final participant in the cohort. Study stopping criteria are applicable throughout the study for all participants. Enrollment will range up to 18 participants depending on whether the maximum tolerated dose (MTD) is reached.

AON1 will be administered at a starting dose of 0.6 mg/kg for Cohort 1A (Part 1) participants (n=3), with an interval of at least 9 days between dosing of participants. Cohort 1A (Part 1) participants will not be dosed at their Week 2 visit. The DMC will review each participant's safety data through Week 2 and make a recommendation to escalate the participant to 1.5 mg/kg in the absence of DLTs (or expand the cohort to 6 participants if there is a DLT). Cohort 1A (Part 1) participants will receive 1.5 mg/kg AON1 at Week 3, after which the DMC will review each participant's safety data through Week 4 and make a recommendation to escalate the participant to 3 mg/kg in the absence of DLTs (or expand the cohort to 6 participants if there is a DLT). Cohort 1A (Part 1) participants will not be dosed at their Week 4 visit. Cohort 1A (Part 1) participants will receive 3 mg/kg AON1 (single dose) at Week 5, after which the DMC will review each participant's safety data through Week 6 and make a recommendation to escalate the participant to 6 mg/kg in the absence of DLTs (or expand the cohort to 6 participants if there is a DLT). Cohort 1A (Part 1) participants will not be dosed at their Week 6 visit. Cohort 1A (Part 1) participants will receive 6 mg/kg AON1 (single dose) at Week 7, after which the DMC will review each participant's safety data through Week 8 and make a recommendation to initiate Part 2 with weekly dosing at 6 mg/kg in the absence of DLTs (or expand the cohort to 6 participants in Part 1 if there is a DLT). Cohort 1A (Part 1) participants will not be dosed at their Week 8 visit.

If a DLT occurs among Cohort 1A participants, an additional 3 participants will be enrolled at the dose level at which the DLT occurred and then individually dose escalated according to the dose escalation rules outlined above for Cohort 1A (Part 1).

The visit after Week 8 will be the start of Part 2 for Cohort 1A participants, designated the Baseline (Week 1) visit for the 6 mg/kg QW dose level. Dosing of Cohort 1A (Part 2) participants at 6 mg/kg QW will be spaced at least 9 days apart. After all 3 Cohort 1A participants have received 3 doses at 6 mg/kg QW in Part 2 and the third participant completes Week 4 assessments, the DMC will review available safety data and, if there are no DLTs, make a recommendation to open Cohort 1B for dosing at 6 mg/kg QW.

Dosing of Cohort 1B participants at 6 mg/kg QW will be spaced at least 9 days apart. After all 3 Cohort 1B participants have received 3 doses at 6 mg/kg QW in Part 2 and the last participant completes Week 4 assessments, the DMC will review available safety data for all 6 Cohort 1 participants and, if there is no more than 1 DLT, make a recommendation to open Cohort 2 for dosing at 9 mg/kg QW.

AON1 will be administered at 9 mg/kg QW for Cohort 2 participants (n=6), with an interval of at least 9 days between participants. After all 6 Cohort 2 participants have received 3 doses and the last participant completes Week 4 assessments, the DMC will review available safety data and, if there is no more than 1 DLT, make a recommendation to open Cohort 3 for dosing at 12 mg/kg QW.

AON1 will be administered at 12 mg/kg QW for all Cohort 3 participants (n=6), with an interval of at least 9 days between participants.

If 2 or more participants experience a DLT in a given cohort, the MTD will have been exceeded and further dose escalation will not be pursued. The DMC, after data review, may recommend expanding the prior cohort, selecting a lower intermediate dose, or halting the study.

The primary safety assessment endpoints are listed in the “Objectives and Endpoints” table above. The secondary objectives of the study include an evaluation of the plasma and urine PK and muscle distribution of AON1. All participants will have a muscle biopsy at Screening and Week 13 (Cohort 1A) or Week 25 (Cohorts 1B, 2, and 3). After all 6 participants in Cohort 3 (12 mg/kg QW) complete assessments for Week 25, all data will be evaluated in order to determine the dose(s) for a planned long term extension (LTE) study, which will be open to all participants who complete this study and has a planned duration of at least 1 year. Details of the LTE study will be specified in a separate protocol. Once the dose(s) is determined and the LTE study is open for enrollment, participants from this study will either transition to the LTE study or have their Study Completion visit 4 weeks after their final dose of AON1. Until the LTE study is open, dosing and assessments will continue in this study.

After the last participant has received his dose at Week 25, the DMC will review all participant data. Based on the results and DMC recommendation, the sponsor will determine the dose(s) for the LTE study. A participant is considered to have completed the study if he has completed all scheduled visits through the sponsor's determination of the dose(s) for the LTE. Following health authority clearance or approval, and after a participant's site receives IRB/EC approval for the LTE, the participant may begin dosing in the LTE. Study completion occurs on the date of the last visit prior to the participant enrolling into the LTE. During the LTE study participants will receive AON1 at 12 mg/kg or the highest tolerated dose(s) as determined during this study.

### Example 11

### Evaluation of AON1 26-Week Toxicity Study in Male CD-1 Mice

Male CD-1 mice (10 or 15/group (main toxicity study) and up to 5/group of main toxicity animals in the 0 and 18 mg/kg dose groups euthanized after a 13-week recovery phase; 4 (vehicle control) and 19/group (toxicokinetic (TK))) were dosed via slow bolus IV injection with 0 (vehicle: 0.9% NaCl Injection, USP), 6, 12, or 18 mg/kg twice weekly AON1 for a total duration of 26 weeks. Blood was collected on Days 1 and 176 at 0 (within 2 minutes postdose), 0.33, 1, 3, 8, and 24 hours postdose for bioanalyses. The following parameters were evaluated: mortality, clinical signs, body weights, food consumption, ophthalmic examinations, clinical pathology (ie, clinical chemistry, hematology, and coagulation), gross necropsy, organ weights, and histopathology.

### Bioanalysis and Tissue Distribution

Plasma exposure (based on Cₘₐₓ and AUC₀₋₂₄) increased with increasing dose in a generally dose-proportional manner. No accumulation was observed in male mice following multiple doses of AON1.

Mean concentrations of AON1 in male mouse tissues were measurable on Day 178 in all treated groups. The mean concentrations generally increased with an increase in dose from 6 to 18 mg/kg/dose. The rank order of mean concentrations of AON1 across dose levels were: liver, kidney, heart, gastrocnemius, and bicep femoris.

Due to the mild severity of pathology findings and the lack of impact on the health or well-being of animals administered up to 18 mg/kg/dose twice weekly, the no observed adverse effect level (NOAEL) was18 mg/kg/dose twice weekly, which corresponded to mean maximum observed concentrations (Cₘₐₓ) and area under the concentration-time curves (AUC₀₋₂₄) values of 37,700 nmol/L and 14,700 h*nmol/L, respectively, on Day 176 of the dosing phase.

### Example 12

### Cytochrome P450 Inhibition and Induction

The potential for AON1 to inhibit CYP enzymes was evaluated in human liver microsomes incubated with 0.1 to 100 μM AON1. Validity of the assay was confirmed with positive controls (i.e., reference compounds) for each isoenzyme. The IC₅₀ value for direct inhibition of CYP1A2 was 7.85 μM, while the IC₅₀ value for direct inhibition of CYP2C8 and CYP2C19 was >100 μM. After accounting for microsomal protein binding, the unbound IC₅₀ values for direct inhibition of CYP1A2, CYP2C8, and CYP2C19 were 0.09, >1.27, and >1.08 μM, respectively, which were lower than the predicted human plasma Cₘₐₓ of 18 μM at the maximum dose of 18 mg/kg. Plasma protein binding of AON1 was attempted and could not be evaluated due to high levels of non-specific binding to filtration devices. There was no direct inhibition of CYP2B6, CYP2C9, CYP2D6, CYP3A4/5 (substrate=testosterone or midazolam). Based on literature, phosphorothioate-based oligonucleotide inhibition of CYP enzymes was testing system dependent; inhibition of CYP enzymes by phosphorothioate-based oligonucleotides was observed in human liver microsomes and not in cryopreserved human hepatocytes (Kazmi Drug Metab Dispos. 2018 August;46(8):1066-1074. Available at: https://dmd.aspetjournals.org/content/dmd/46/8/1066.full.pdf). Cryopreserved human hepatocytes provide a more clinically relevant inhibitory profile for use in in vitro to in vivo extrapolation of drug-drug interactions for phosphorothioate-based oligonucleotide inhibition of CYP enzymes. Given AON1 is also a phosphorothioate-based oligonucleotide, microsomes may not be representative of in vivo conditions.

To that end, a further investigation using cryopreserved human hepatocytes was conducted and demonstrated AON1 is not a direct or time-dependent inhibitor of CYP1A2, CYP2B6, CYP2C8, CYP2C9, CYP2C19, CYP2D6 or CYP3A4/5 with IC₅₀ values >100 μM for all CYPs evaluated.

The potential to induce CYP enzymes was evaluated in hepatocytes incubated with 100 μM AON1. Validity of the assay was confirmed with positive controls (i.e., reference compounds) for each isoenzyme. The in vitro assay for CYP induction indicated CYP1A2, CYP2B6, and CYP3A4 were not induced by AON1 at the transcription level.

### Example 13

### Evaluation of AON1 in Male Cynomolgus Monkeys Treated for 13 Weeks

Male cynomolgus monkeys (3/group (main toxicity study); 2/group for 0, 12, and 18 mg/kg QW (4-week recovery)) were dosed via IV infusion (60 minutes) with 0 (vehicle: NaCl Injection, USP), 6, 12, or 18 mg/kg QW AON1 for 13 weeks. Blood was collected on Days 1 and 85 at predose, and 0.5, 1, 2, 3, 6, 8, and 24 hours postdose for bioanalyses and at predose, 1, 3, 6, and 24 hours postdose for complement activation analysis. A predose sample was also collected on Day 78. The following parameters were evaluated: mortality, clinical signs, body weight, food consumption, AON1 levels in plasma and tissues (gastrocnemius, heart, biceps femoris, liver, and kidney), percent exon skipping in tissues (gastrocnemius, heart, biceps femoris, liver, and kidney), clinical pathology (clinical chemistry, hematology, urinalysis, urine chemistry, and urine biomarkers), complement activation indicators (Bb, C3a, and sC5b-9), ophthalmology, ECG, organ weights, and histopathology.

Exon skipping in tissues was measured by RT-ddPCR. The method quantified the number of non-skipped DMD (dystrophin) transcripts, and the number of exon 51-skipped DMD transcripts; and the percent exon skipping (% skip) was calculated. The percent exon 51 skip varied from 1.7% to 14.4% and generally increased with dose.

This disclosure is not to be limited in scope by the embodiments disclosed in the examples which are intended as single illustrations of individual aspects, and any equivalents are within the scope of this disclosure. Various modifications in addition to those shown and described herein will become apparent to those skilled in the art from the foregoing description. Such modifications are intended to fall within the scope of the appended claims.

Various references such as patents, patent applications, and publications are cited herein, the disclosures of which are hereby incorporated by reference herein in their entireties.

What is claimed is:

1. A method of treating a subject having DMD or delaying the onset of DMD in a subject, comprising administering to the subject AON1 at a dose of 0.4 mg/kg, 0.6 mg/kg, 0.8 mg/kg, 1.5 mg/kg, 3 mg/kg, 6 mg/kg, 9 mg/kg, 12 mg/kg or 18 mg/kg, wherein the AON1 is administered QW.

2. The method of claim 1, wherein the AON1 is administered QWx15, 16, 17, 18, 19, 21, 22, 23, 24, 25, 26, 27, 28, 29, 30, 31, 32, 33, 34 or 35W.

3. The method of claim 1, wherein the AON1 is administered QWx24W or QWx25W.

4. The method of claim 3, wherein the AON1 is administered QWx25W.

5. The method of claim 3, wherein the AON1 is administered QWx24W.

6. The method of claim 1, wherein the AON1 is administered at a dose of 3 mg/kg.

7. The method of claim 1, wherein the AON1 is administered at a dose of 6 mg/kg.

8. The method of claim 1, wherein the AON1 is administered at a dose of 9 mg/kg.

9. The method of claim 1, wherein the AON1 is administered at a dose of 12 mg/kg.

10. The method of claim 1, wherein the AON1 is administered at a dose of 18 mg/kg.

11. The method of claim 1, that is a method of treating DMD in a subject.

12. The method of claim 1, that is a method of delaying the onset of DMD in a subject.

13. The method of claim 1, further comprising administering to the subject a second active agent.

14. The method of claim 13, wherein the second active agent is eteplirsen, casimersen, golodirsen, viltolarsen, SRP-5051 (Sarepta Therapeutics), a corticosteroid such as deflazacort, a gene therapy (e.g., SRP-9001, GALGT2 or GNT 0004 (Sarepta Therapeutics)), gene editing (e.g., CRISPR/CAS9 (Sarepta Therapeutics)) or a cellular therapy (e.g., CAP-1002 (Capricor Therapeutics/Nippon Shinyaku Co. Ltd.)).


SEQUENCE LISTING (2 sequences)

SEQ ID NO: 1 - RNA, 18 nt, synthetic construct
  source 1..18: mol_type = other RNA
ggtaagttctgtccaagc

SEQ ID NO: 2 - RNA, 18 nt, synthetic construct
  source 1..18: mol_type = other RNA
  modified_base 9: mod_base = m5c
  modified_base 13: mod_base = m5c
  modified_base 14: mod_base = m5c
  modified_base 18: mod_base = OTHER; note = 2',4'-methyleneoxy-5-methylcytidine
  modified_base 11: mod_base = gm
  modified_base 17: mod_base = gm
  modified_base 1: mod_base = OTHER; note = 2',4'-methyleneoxyguanosine
  modified_base 2: mod_base = OTHER; note = 2',4'-methyleneoxyguanosine
  modified_base 3: mod_base = OTHER; note = 2'-O-methyluridine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2',4'-methyleneoxyguanosine
  modified_base 7: mod_base = OTHER; note = 2'-O-methyluridine
  modified_base 8: mod_base = OTHER; note = 2'-O-methyluridine
  modified_base 10: mod_base = OTHER; note = 2'-O-methyluridine
  modified_base 12: mod_base = OTHER; note = 2'-O-methyluridine
  modified_base 15: mod_base = OTHER; note = 2'-O-methyladenosine
  modified_base 16: mod_base = OTHER; note = 2'-O-methyladenosine
ggtaagttctgtccaagc